SEQUENCE LISTING (3 sequences)

SEQ ID NO: 1 - DNA, 17 bp, Homo sapiens
ctttccgacacctgatt

SEQ ID NO: 2 - DNA, 19 bp, Homo sapiens
tagccaattggggtctttg

SEQ ID NO: 3 - DNA, 700 bp, Homo sapiens
acatggtggtgccagtgacagtctgtgttttggggcaggatagaaactaacctgcttcagttacctgacattctagcaaacttattaggaacacaaacgctcgggtctctgaatgcaccaacctgagcgccggctgattaggcattgtaaagcagtgttctaaaaggaaaggccttcacttaaacagtgctacaacgtgtttgttcagctttctttcatacacaaacttttgccagaaaaggcgttttaagccgagggtaaagattttgtgcgtccaccgttcccatcttcaactctttagaataatagtcatttaagcaccggacctgtcttcagattcttactttgcgacacagctttggccggacttggcttgatctgggctccaggatcggtcccaccacccgggctcggagcggtttgttcctagtggatcagggcgggtgtgttgccggagtcgccttctattggctacactcccggggactggctgggctttccgacacctgattgggcggaacagccctctgtacgccgacatcattggagggcgctggagccagggggcggagcgggttccccaggattcttgaccgggcgcgctagtccgtccgctgagccgggcgcggggcgcaagagcggagctgcgcgagccgctgcggagggaagggctcctagccaattggggtctttgaggcga

(19) United States
(12) Patent Application Publication
(10) Pub. No.: US 2022/0152082 A1
(43) Pub. Date: May 19, 2022

(54) Methods for Cancer Diagnosis, Prognosis or Treatment

(71) Applicant: AnPac Bio-Medical Science Co., Ltd., Tortola (VG)

(72) Inventor: He Yu, Honolulu, HI (US)

(21) Appl. No.: 17/436,920

(22) PCT Filed: Mar. 6, 2020
(86) PCT No.: PCT/US2020/021560

§ 371 Date: Sep. 7, 2021

Related U.S. Application Data
(60) Provisional application No. 62/815,193, filed on Mar. 7, 2019.

(51) Int. Cl. A61K 31/7105; A61P 35/00; C12Q 1/6886

(52) U.S. Cl. CPC A61K 31/7105; A61K 48/00; C12Q 1/6886; A61P 35/00

(57) ABSTRACT

The present invention is generally directed to a method for diagnosis, prognosis, or treatment of a cancer, by detecting or upregulating lncRNA, LINC00472. Particularly, LINC00472 is associated with the prognosis of ER-positive breast cancer and is involved in the interplay between ERα and NF-κB.

### CROSS-REFERENCE TO RELATED APPLICATION

This application claims priority to U.S. Application No. 62/815,193, filed Mar. 7, 2019, the contents of which are incorporated herein by reference in their entirety.

### FIELD OF THE INVENTION

The present invention relates to methods for diagnosis, prognosis, and/or treatment of a cancer, comprising detecting a level of RNA linc00472 in a biological subject and/or regulating the expression of RNA linc00472, to mediate the inhibitory effect of ERα on NF-κB.

### BACKGROUND OF THE INVENTION

Breast cancer is one of the most frequently occurring cancers in the world. Each year, more than 180,000 and 1 million women in the U.S. and worldwide, respectively, are diagnosed with breast cancer. Breast cancer is the leading cause of death for women between ages 50-55, and is the most common non-preventable malignancy in women in the Western Hemisphere. The cancer also evolves substantially over time in patients after they receive chemo or hormonal therapies. These features impose significant challenges to the success of patient treatment.

Status of estrogen receptor (ER) in breast cancer is an important biomarker for predicting disease prognosis and patient's response to adjuvant endocrine therapy. Most breast cancer patients with ER-positive tumors have good prognosis and favorable responses to endocrine therapy, but some may develop endocrine resistance which leads to disease recurrence and tumor metastasis. Multiple mechanisms have been proposed to explain the mechanisms of endocrine resistance in ER-positive patients, one of which is the reciprocal repression of two important transcription factors in breast cancer, ERα and NF-κB. Evidence has suggested that one of the ERα actions is to inhibit the activity of NF-κB and that blocking ER may release its inhibition on NF-κB. NF-κB is a strong signal for inflammatory cytokines and is known to play a crucial role in tumor progression and metastasis. However, how ER represses the activity of NF-κB remains to be elucidated.

Long non-coding RNAs (lncRNAs) are a group of newly discovered RNA transcripts which are transcribed from the DNA templates by RNA polymerase II, but devoid of evident open reading frames and codes for protein translation. Although 75% of human genome are transcribed, only 2% of the genome encodes proteins. Over 90% of the transcripts are not translated, known as non-coding RNA. Conventional research on breast cancer focused largely on proteins and protein-coding genes—with very little attention to non-coding RNAs, especially long non-coding RNAs (lncRNAs). Until recently, some studies indicate that non-coding RNAs may play an important role in cell biology. That said, research on non-coding RNAs in cell biology, particularly in cancer diagnosis, prognosis and/or treatment, is a new trend and is still developing.

Accordingly, it is desirable to provide methods for cancer diagnosis, prognosis and/or treatment, using a lncRNA that is associated with the prognosis of ER-positive breast cancer. It is also desirable to provide methods of inhibiting NF-κB pathway—as well as methods for determining or predicting the resistance of a cancer cell—by using a lncRNA that is involved in the interplay between ERα and NF-κB.

### SUMMARY OF THE INVENTION

The present invention in general relates to methods for diagnosis, prognosis, or treatment of a cancer, methods for determining or predicting the resistance of a cancer cell, and methods for inhibiting an activity of NF-κB pathway in a biological subject. Particularly, the methods described in the present invention use a lncRNA that is associated with the prognosis of ER-positive breast cancer and/or involved in the interplay between ERα and NF-κB.

One key component of the present invention is lncRNA, LINC00472, which appears to be a tumor suppressor in breast cancer and its expression is associated with favorable prognosis. As further described in the present application, experiments reveal upregulation of LINC00472 expression by ERα and suppression of NF-κB activation by LINC00472. High expression of LINC00472 is found to exist in ER-positive tumor, and the lncRNA is associated with favorable prognosis of ER-positive patients. Also, it is discovered that LINC00472 appears to play an important role in mediating the inhibitory effect of ERα on NF-κB, which suggests that suppressing ERα by endocrine therapy may release this inhibition, leading to tumor progression and treatment resistance.

According to the present invention, the analysis of LINC00472 transcriptome revealed ERα regulation of LINC00472 expression, and an ERα-binding site in the LINC00472 promoter was identified. In vitro experiments also confirmed up-regulation of LINC00472 expression by ERα. Transcriptome and metabolome of LINC00472 overexpression further indicated a possible interaction between LINC00472 and NF-κB, which was confirmed in cell experiments, showing suppression of phosphorylation in p65 and IκBα by LINC00472. Further experiments also demonstrated that suppression of NF-κB by ERα was mediated through LINC00472. High LINC00472 expression inhibited tumor growth both in vitro and in vivo and suppressed aggressive tumor cell behaviors in vitro. Knockdown of LINC00472 expression could reverse the cell aggressive behaviors in vitro. Tamoxifen treatment of ER-positive tumor cells inhibited ERα and LINC00472 expression, and increased phosphorylation of p65 and IκBα Meta-analysis showed that LINC00472 expression were higher in ER-positive than in ER-negative tumors and high expression was associated with better survival in patients with ER-positive tumor.

One aspect of the present invention relates to a method of determining or predicting the resistance of a cancer cell (e.g., a breast cancer cell) in a biological subject to a treatment, comprising: (i) detecting an expression of RNA linc00472 in a sample obtained from the biological subject; (ii) comparing the detected expression with a control; and (iii) determining or predicting the resistance of the cancer to the endocrine therapy based on the comparison.

In some embodiments, the treatment inhibits ERα pathway in the biological subject and/or elevates the activity of NF-κB pathway in the biological subject. For instance, the treatment may be an endocrine therapy.

In some embodiments, a higher detected level of RNA linc00472 as compared to the control indicates a better relapse-free survivor and/or overall survival.

Another aspect of the present invention relates to a kit for diagnosing or treating a cancer or predisposition thereto in a biological subject, or determining the resistance of the cancer to a treatment, comprising (i) a biomarker that detects the level of RNA linc00472 in a sample obtained from the biological subject; and (ii) a control, to be compared with the detected level.

In some embodiments, a higher detected level of RNA linc00472 as compared to the control indicates the presence of the cancer or predisposition thereto. In some embodiments, a higher detected level of RNA linc00472 as compared to the control indicates a better relapse-free survivor and/or overall survival than a predetermined survivor rate.

In some embodiments, the cancer is estrogen receptor-positive, preferably a breast cancer. In some further embodiments, the treatment is an endocrine therapy.

A further aspect of the invention relates to a method of treating a cancer or predisposition thereto, in a biological subject, comprising administration of an effective amount of a NF-κB pathway inhibitor.

In some embodiments, the NF-κB pathway inhibitor upregulates a level of RNA linc00472 in the biological subject. In some embodiments, the NF-κB pathway inhibitor comprises a linc00472-expressing plasmid or vector.

In another aspect, the present invention provides a method of inhibiting an activity of NF-κB pathway in a biological subject, comprising a step of upregulating a level of RNA linc00472 in the biological subject, e.g., by administrating an effective amount of a composition that upregulates the level of RNA linc00472, to the biological subject.

In some embodiments, the composition comprises a linc00472-expressing plasmid. As such, the cells in the biological subject may contact, and transfected with, a linc00472-expressing plasmid, thereby resulting the overexpression of linc000472.

In some embodiments, the composition further comprises a pharmaceutically acceptable carrier. In some embodiments, the composition comprises a transfection agent to facilitate the delivery of RNA linc00472 to the cells.

In still a further aspect, the present invention provides a method of preventing the resistance of a cancer cell in a biological subject to a treatment (e.g., an endocrine therapy), comprising administration of an effective amount of a compound that upregulates the level of RNA linc00472 in a sample.

In some embodiments, the compound comprises a linc00472-expressing plasmid or vector. Such a method allows the cancer cell to contact with a linc00472-expressing plasmid or vector.

### BRIEF DESCRIPTIONS OF THE DRAWINGS

FIGS. 1A-1N, 2, 3A-3F, and 4 illustrate the association of LINC00472 expression with breast cancer survival.

FIGS. 1A-1N illustrate Kaplan-Meier relapse-free survival curves (RFS) by high and low expression of LINC00472.

FIG. 2 illustrates a meta-analysis of hazards ratios, which shows a reduced risk for relapse in patients with high LINC00472 expression compared to those with low expression (HR=0.574; 95% CI: 0.504-0.654).

FIGS. 3A-3F illustrate Kaplan-Meier overall survival curves (OS) by high and low expression of LINC00472.

FIG. 4 illustrates a meta-analysis of hazards ratios, which shows a reduced risk for death in patients with high LINC00472 expression compared to those with low expression (HR=0.562; 95% CI: 0.370-0.853).

FIGS. 5A-5B, 6A-6D, 7A-7D, 8A-8D, 9A-9B, and 10 illustrate LINC00472 suppression of tumor cell proliferation, migration, invasion, and colony formation.

FIG. 5A illustrates LINC00472 expression in the tested breast cancer cell lines. Comparisons were made between MCF-7 and other cell lines. *=P<0.05, ***=P<0.0001.

FIG. 5B illustrates LINC00472 expression in MB231 and Hs578T cells, after a LINC00472-expressing plasmid was transfected into the cells (pool: MB231-Linc00472 and Hs578T-Linc00472; single clone: MB231-Linc00472-1 and Hs578T-Linc00472-1), in comparison to those transfected with an empty vector (mock) (pool: MB231-Mock and Hs578T-Mock); single clone: MB231-Mock-1 and Hs578T-Mock-1). β-actin was used as reference in expression analysis, and comparisons were made between cells transfected with a LINC00472 or Mock plasmid. ***=P<0.0001.

FIGS. 6A-6D illustrate cell proliferation in MB231 and Hs578T cells with LINC00472 overexpression compared to those without overexpression. ***=P<0.0001.

FIGS. 7A-7D illustrate migration in MB231 and Hs578T cells when LINC00472 was overexpressed compared to those without overexpression. ***=P<0.0001.

FIGS. 8A-8D illustrate invasion in MB231 and Hs578T cells when LINC00472 was overexpressed compared to those without overexpression. ***=P<0.0001.

FIGS. 9A-9B illustrate colony formation in MB231 and Hs578T cells with LINC00472 overexpression compared to those without overexpression. **=P<0.001, ***=P<0.0001.

FIG. 10 illustrates a flow cytometry analysis of cell cycle, which shows fewer MB231 and Hs578T cells in G2 phase when LINC00472 is overexpressed compared to those without overexpression. *=P<0.05, ***=P<0.0001.

FIGS. 11A-11E illustrate LINC00472 inhibition of breast tumor growth in a xenograft mouse model

FIG. 11A is a picture showing 7 BALB/c female nude mice 25 weeks after injection of MB231 cells in mammary fat pads, with cells with LINC00472 overexpression on the left and those without overexpression on the right.

FIG. 11B is a picture showing 7 pairs of tumors dissected from the left (top line) and right side (bottom line) of the animals in Panel A, which shows that tumors on the top were smaller than those on the bottom; and no tumors were detected in the left fat pad of two mice on the right.

FIG. 11C illustrates average tumor volumes (mm3) measured at each time post injection (days). ***=P<0.0001.

FIG. 11D shows a much higher level of LINC00472 levels in the tumors. ***=P<0.0001.

FIG. 11E illustrates H&E staining of tumor tissues, which shows that tumors with LINC00472 overexpression have fewer cells and less malignant morphology than those without overexpression.

FIGS. 12A-12G illustrate an analysis of transcriptome, metabolome and NF-κB inactivation associated with LINC00472 overexpression.

FIGS. 12A-12B illustrate Venn diagram showing the numbers of genes up- and down-regulated in MB231 and Hs578T cells due to LINC00472 overexpression (two pools and two single clones in each cell line, and a total of eight samples).

FIGS. 12C-12D illustrate changes in expression of one up-(DXO) and two down-regulated (Linc01061, MALAT1) genes verified by qRT-PCR in the cell lines. ***=P<0.0001.

FIG. 12E illustrates a heatmap showing differentially expressed genes in MB231 and Hs578T cells due to LINC00472 overexpression; among which five top canonical pathways are predicted by Ingenuity Pathway Analysis (IPA) based on differentially expressed genes in MB231 and Hs578T.

FIG. 12F illustrates a heatmap showing differentially detected metabolites in MB231 and Hs578T cells due to LINC00472 overexpression; among which five top canonical pathways are predicted by IPA based on differentially detected metabolites in MB231 and Hs578T.

FIG. 12G illustrates Western blot analysis showing reduced phosphorylation of p65 (p-P65) and IκBα in MB231 and Hs578T cells with LINC00472 overexpression.

FIGS. 13A-13K illustrate ERα upregulation of LINC00472 expression

FIG. 13A illustrates IPA that predicts 8 possible molecules involved in the regulation of LINC00472 expression in both MB231 and Hs578T cells.

FIG. 13B illustrates Western blot analysis showing high ERα expression in 293T cells after an ESR1-expressing plasmid (pCMV-ESR1) is transfected into the cells.

FIG. 13C illustrates Luciferase reporter assay, which shows the interaction between ERα and the LINC00472 promoter predicted by PROMO in 293T after the cells are co-transfected with the ESR1 plasmid (pCMV-ESR1) and a luciferase report, linked either to a wild type of LINC00472 promoter (pGL4-Linc00472-wt) or a mutant (pGL4-Linc00472-mut).

FIG. 13D illustrates ChIP-qPCR analysis showing the interaction between ERα and the LINC00472 promoter. ***=P<0.0001.

FIG. 13E illustrates Western blot analysis showing increased ERα expression in Hs578T after the cells are transfected with the ESR1 plasmid (pCMV-ESR1).

FIG. 13F illustrates qRT-PCR analysis showing increased LINC00472 expression after 72-hour incubation of Hs578T cells with ERα overexpression. ***=P<0.0001.

FIG. 13G illustrates Western blot analysis, which shows increased ERα expression in MB231 after the cells are transfected with the ESR1 plasmid (pCMV-ESR1).

FIG. 13H illustrates qRT-PCR analysis which shows increased LINC00472 expression after 72-hour incubation of MB231 cells with ERα overexpression. ***=P<0.0001.

FIG. 13I illustrates Western blot analysis, which shows ERα suppression of p65 (p-p65) and IκBα (p-IκBα) phosphorylation in Hs578T and MB231 after the cells are transfected with the ESR1 plasmid (pCMV-ESR1).

FIG. 13J illustrates qRT-PCR analysis, which shows significant reduction of LINC00472 expression in Hs578T and MB231 after LINC00472 knockdown by siRNA (siLinc00472). ***=P<0.0001.

FIG. 13K illustrates Western blot analysis, which shows that LINC00472 knockdown in Hs578T and MB231 abolished ERα suppression of p65 and IκBα phosphorylation.

FIGS. 14A-14G illustrate the activation of NF-κB by LINC00472 knockdown and downregulation of LINC00472 by tamoxifen in T47D cells.

FIG. 14A illustrates qRT-PCR analysis, which shows reduction in LINC00472 expression in T47D after siRNA knockdown of LINC00472 (siLinc00472). ***=P<0.0001.

FIG. 14B illustrates Western blot analysis, which shows increased phosphorylation of p65 and IκBα in T47D after siRNA knockdown of LINC00472.

FIG. 14C illustrates increased cell proliferation in T47D after treatment of LINC00472 siRNA (siLinc00472), as compared to control siRNA (siCtrl).

FIG. 14D illustrates increased cell migration in T47D after treatment of LINC00472 siRNA (siLinc00472), as compared to control siRNA (siCtrl).

FIG. 14E illustrates increased cell invasion in T47D after treatment of LINC00472 siRNA (siLinc00472), as compared to control siRNA (siCtrl).

FIG. 14F illustrates Western blot analysis showing increased phosphorylation of p65 and IκBα and decreased expression of ERα in T47D cells after 4-hydroxytamoxifen treatment for 8 days.

FIG. 14G illustrates qRT-PCR analysis showing decreased expression of LINC00472 in T47D cells after 4-hydroxytamoxifen treatment for 8 days. ***=P<0.0001.

FIGS. 15A-15P illustrate the association of LINC00472 expression with breast cancer survival in patients with ER-positive tumors.

FIGS. 15A-15K show Kaplan-Meier relapse-free survival curves (RFS) by high and low expression of LINC00472 in ER-positive tumors.

FIG. 15L illustrates a meta-analysis of hazards ratios showing a reduced risk for relapse in ER-positive patients with high LINC00472 expression compared to those with low expression (HR=0.530; 95% CI: 0.445-0.631).

FIGS. 15M-15O show Kaplan-Meier overall survival curves (OS) by high and low expression of LINC00472 in ER-positive tumors.

FIG. 15P illustrates a meta-analysis of hazards ratios, showing a reduced risk for death in ER-positive patients with high LINC00472 expression compared to those with low expression (HR=0.510; 95% CI: 0.332-0.782).

### DETAILED DESCRIPTION OF THE INVENTION

The present invention in general relates to the detection or regulation of a lncRNA, for methods of cancer diagnosis, prognosis, or treatment; methods for determining or predicting the resistance of a cancer cell; and methods for inhibiting an activity of NF-κB pathway in a biological subject. Particularly, the lncRNA is LINC00472, which appears to be associated with the prognosis of ER-positive breast cancer and involved in the interplay between ERα and NF-κB.

As the key component of the present invention, LINC00472 is found to play an important role in breast cancer as its expression is upregulated by ERα and high expression is associated with ER-positive tumors and favorable prognosis. More importantly, LINC00472 suppresses the activity of NF-κB, and the inhibition of NF-κB by ERα is mediated through LINC00472. Endocrine treatment reduces the activity of ER which subsequently suppresses LINC00472, resulting in the release of its inhibition on NF-κB. As NF-κB is an important signal in promoting tumor growth and metastasis, the involvement of LINC00472 in endocrine therapy-induced suppression of ERα and activation of NF-κB may serve as a new molecular pathway underlying the mechanism of endocrine resistance. Therefore, LINC00472 may also play an important role in providing a novel strategy to overcome endocrine resistance.

Accordingly, one aspect of the present invention provides a method of determining or predicting the resistance of a cancer cell (e.g., a breast cancer) in a biological subject to a treatment (e.g., an endocrine terapy), which may comprise at least the following steps: (i) detecting an expression of RNA linc00472 in a sample obtained from the biological subject; (ii) comparing the detected expression with a control; and (iii) determining or predicting the resistance of the cancer to the endocrine therapy based on the comparison. In some embodiments, the treatment inhibits ERα pathway in the biological subject and/or elevates the activity of NF-κB pathway in the biological subject. Particularly, a higher detected level of RNA linc00472 as compared to the control may indicate a better relapse-free survivor and/or overall survival.

Another aspect of the present invention provides a kit for diagnosing or treating a cancer or predisposition thereto in a biological subject, or determining the resistance of the cancer to a treatment, comprising (i) a biomarker that detects the level of RNA linc00472 in a sample obtained from the biological subject; and (ii) a control, to be compared with the detected level. In some embodiments, a higher detected level of RNA linc00472 as compared to the control indicates the presence of the cancer or predisposition thereto. In some embodiments, a higher detected level of RNA linc00472 as compared to the control indicates a better relapse-free survivor and/or overall survival than a predetermined survivor rate.

In some embodiments, the cancer is estrogen receptor-positive. In some preferred embodiments, the cancer is breast cancer.

A further aspect of the present invention provides a method of treating a cancer or predisposition thereto, in a biological subject, comprising administration of an effective amount of a NF-κB pathway inhibitor. In some embodiments, the NF-κB pathway inhibitor upregulates a level of RNA linc00472 in the biological subject. For example, the NF-κB pathway inhibitor comprises a linc00472-expressing plasmid or vector.

Still a further aspect of the invention relates to a method of inhibiting an activity of NF-κB pathway in a biological subject, by upregulating a level of RNA linc00472 in the biological subject. In some embodiments, an effective amount of a composition that upregulates the level of RNA linc00472 may be administrated to the biological subject. For instance, the cells in the biological subject may contact, and transfected with, a linc00472-expressing plasmid, thereby resulting the overexpression of linc000472. In some embodiments, the composition further comprises a pharmaceutically acceptable carrier, and/or a transfection agent to facilitate the delivery of RNA linc00472 to the cells.

In still another aspect, the present invention provides a method of preventing the resistance of a cancer cell in a biological subject to a treatment (e.g., an endocrine therapy), comprising administration of an effective amount of a compound that upregulates the level of RNA linc00472 in a sample. In some embodiments, the method comprises contacting the cancer cell with a linc00472-expressing plasmid or vector.

To demonstrate the association and the mechanisms of LINC00472 in the tumor, the molecular targets and regulation of LINC00472 in breast cancer cells were studied, and patient information from multiple clinical datasets were analyzed. Additionally, breast cancer cells transfected with a LINC00472-expressing plasmid were analyzed for their transcriptomes and metabolomes, and the identified molecular target and modulator were validated in a series of in vitro experiments. Cell behaviors with LINC00472 overexpression were evaluated in vitro and in vivo, and with LINC00472 knockdown assessed in vitro. Meta-analysis was performed with multiple online datasets including our own study to demonstrate the associations of LINC00472 with ER status and disease outcomes.

### Materials and Methods

### Study Patients

525 patients were recruited with primary breast cancer for study. These patients were identified in two hospitals in Turin, Italy, including 348 patients in S. Anna Hospital enrolled between January 1998 and July 1999, and 177 patients in Mauriziano Hospital recruited between October 1996 and August 2012. The average age of patients at surgery was 58 years, and the age range was between 23 and 89 years. Information on disease stage, tumor grade, hormone receptor status, follow-up time and survival outcomes was extracted from patient medical records. The study was approved by the ethic review committees at the hospitals.

### Online Datasets

Data on LINC00472 expression in breast cancer were extracted from the NCBI GEO database. A total of 15 datasets with at least 100 patients in each were identified from the database, of which 13 had relapse-free survival information (GSE19615, GSE42568, GSE1456, GSE53031, GSE7390, GSE11121, GSE22219, GSE3494, GSE21653, GSE4922, GSE31448, GSE2034, GSE25066), and 5 had overall survival data (GSE42568, GSE16446, GSE1458, GSE7390, GSE20685). Of these datasets, 10 and 2, respectively, had information on both survival and ER status. The search was updated in June 2017.

### RNA Extraction and LINC00472 Measurement

Total RNA was extracted from fresh-frozen tumor samples and cultured tumor cells using the Allprep DNA/RNA kit (Qiagen). RNA samples were reverse-transcribed (RT) to cDNA using the cDNA Reverse Transcription kit (LifeTech), and analyzed for LINC00472 expression with real-time PCR (qPCR).

### Western Blot Analysis

Cell lysates were prepared in a lysis buffer purchased from Roche. The lysates containing 40-60 μg proteins were analyzed with SDS-PAGE under a denaturing condition and the resulting gels were transferred to polyvinylidene difluoride (PVDF) membranes (Millipore). The membranes were blocked with 5% non-fat milk for 45 minutes, and then incubated with a primary antibody followed by a secondary antibody. The signals were detected by an enhanced chemiluminescence system (ECL) following the manufacturer's manual (Pierce). Antibodies used for analysis, including anti-Phospho-NF-κB p65 (Ser536) (#3033), anti-NF-κB p65 (#8242), anti-Phospho-IκBα (Ser32) (#2895), anti-IκBα (#9242) and anti-ERα (#8644), were purchased from Cell Signaling Technology, and anti-β-actin (A2228) was from Sigma-Aldrich.

### Cell Culture and Plasmid Transfection

Breast cancer cell lines, MCF-7, T47D, MDA-MB-231 (MB231) and Hs578T, were obtained as part of the NCI-60 DTP Human Tumor Cell Screening Panel. SKBR3, ZR-75-1, and HEK-293T (293T) cells were purchased from the American Type Culture Collection. Cells were cultured according to the manufacturer's instruction. A LINC00472 transcript (2933 bp, NR 026807.1) was assembled and inserted into a lentiviral vector, pCDH-EF1-MCS-pA-PGK-copGFP-T2APuro (pCDH), as previously described (13). The sequence of the insert was confirmed. MB231 and Hs578T cells were transfected with the LINC00472 plasmid or an empty plasmid (pCDH vector only) named mock using the Lipofectamine 3000 reagent (Thermo Fisher Scientific) following the manufacturer's protocol. Cells with stable expression of LINC00472 were selected through puromycin screening (Thermo Fisher Scientific). To maintain stable cell pool, puromycin was added into culture medium, and the puromycin-containing culture medium was replaced every 3 days. A single cell clone was also generated from the stable cell pool through the limiting dilution cloning.

### Cell Proliferation, Migration, and Invasion

Cell proliferation, migration and invasion were analyzed as previously described. Briefly, for cell proliferation, the cells were seeded onto 96-well plates at 3×103 cells per well. After 2 hours of incubation with the WST-1 cell proliferation reagent (Roche Diagnostics GmbH), cell concentrations were measured at 0, 24, 48 and 72 hours of culture with Optical Density (OD) at 450 nm wavelength using a microplate spectrophotometer (Biotek Synergy 2). Cell migration and invasion assays were performed using the Costar Transwell permeable polycarbonate supports (8.0 μm pores) in 24-well plates (Corning Inc.). Cells at a concentration of 1×104 per well were seeded onto the upper chambers of the Transwell permeable supports coated with 1 mg/ml growth factor-reduced Matrigel matrix for invasion assay and without the Matrigel coating for migration assay (BD Pharmingen). The lower chambers were filled with 600 μl complete culture medium. Cells migrating to the lower chambers were stained with HEME 3Solution (Fisher Diagnostics) after 36 hours of incubation. All the assay results were measured in triplicate, and each assay was repeated 3 times.

### Anchorage Independent Assay

Colony formation assay was performed as follows. Cells at a concentration of 1×103 per well were seeded on 0.3% agarose overlaid onto solidified 0.6% agarose in RPMI1640 with 10% FBS in a 6-well plate. Culture medium (200 μl) containing puromycin was added in each well every three days. After 5 weeks, colonies were counted in 5 selected fields from 3 representative wells using the Bid-Rad colony counter. The assay was repeated 3 times.

### Flow Cytometry Analysis of Cell Cycle

Cells, harvested after 48 hours of incubation and washed twice with PBS, were fixed in 70% ice-cold ethanol and stained with propidium iodide (BD Biosciences) at a concentration of 1×106. Cell populations in different cell cycles were analyzed using the BD Accuri C6 flow cytometer (BD Biosciences). The analysis was performed in triplicate for each experiment, and the experiment was repeated 3 times.

### Tumor Xenograft Model

Seven 5-week old BALB/c female nude mice, SPF grade, were purchased from Shanghai SLAC Laboratory Animal Co., Ltd. for tumor xenograft experiment. The mice were injected with 100 μl of 5×106 MB231 cells (50 μl cell solution mixed with 50 μl Matrigel), and the injections were made in the inguinal mammary fat pad with mock cells on the right and LINC00472 overexpression cells on the left. The mice were monitored for 25 days after injection, and body weight and tumor size were measured every 2-3 days after the first two weeks of injection. Animal procedures were performed according to a study protocol that was approved by the university's Animal Care and Use Committee.

siRNA Knockdown

Breast cancer cells were transfected with Lincode Human LINC00472 (79940) siRNA-SMART pool (D-001320-10-05) or Lincode Non-targeting Pool (D-001320-10-05), negative control from Dharmacon, following the manufacturer's protocol for Lipofectamine RNAiMAX (Thermo Fisher Scientific). The method of transfection was known in the art. Cell lysates were prepared for analysis after 36 hours of incubation of the transfected cells.

### Luciferase Reporter Assay

A plasmid (pCMV-ESR1) containing the full-length human ESR1 (NM_000125, #RC213277) was purchased from Origene Technologies. pGL4.27 [luc2P/minP/Hygro] vector was purchased from Promega. Synthesizing and inserting the wild and mutant LINC00472 promoter sequences into the pGL4.27 vector to make pGL4.27-linc00472-wt and pGL4.27-linc00472-mut plasmids were completed and verified by GENEWIZ LLC. HEK293T cells were first transiently transfected with pCMV-ESR1 using the Lipofectamine 3000 reagent (Themo Fisher Scientific). After 36 hours of incubation, the plasmid of pGL4.27-linc00472-wt or pGL4.27-linc00472-mut, together with the Renilla reporter vector, were transfected into the ERS1-expressing cells. Renilla and firefly luciferase activities were measured using the Dual-Luciferase kit (Promega), following the manufacturer's protocols. The results were normalized with the Renilla reporter for transfection efficiency. Each assay was performed in triplicate, and the experiment was repeated 3 times.

### Chromatin Immunoprecipitation (ChIP)-qPCR Assay

ChIP assay was performed using a Chromatin Immunoprecipitation (ChIP) Assay kit (EMD Millipore). After 48-hour incubation of cells transfected with pCMV-ESR1, formaldehyde was added directly to the culture medium at a final concentration of 1% to crosslink histones and DNA. About 200 μl cell lysates were sonicated to shear DNA into lengths between 200 and 1,000 base pairs. Sonicated nuclear fractions were incubated overnight at 4° C. with a rabbit polyclonal antibody against ERα (#8644T from Cell Signaling Technology) or a rabbit polyclonal antibody against IgG (#12-370 from EMD Millipore) as a control. After that, 60 μl of Protein A Agarose/Salmon Sperm DNA (50% Slurry) were added for another hour of incubation at 4° C. and then the antibody/histone complex were collected. The complex was washed and eluted with a buffer supplied in the kit. The eluted histone complex was mixed with 20 μl of 5 M NaCl and heated at 65° C. for 4 hours to break the histone-DNA crosslink. Then, 10 μl of 0.5 M EDTA, 20 μl of 1 M Tris-HCl, pH 6.5, and 2 μl of 10 mg/ml Proteinase K were added and the mixtures were incubated for 1 hour at 45° C. DNA in the samples were isolated through phenol/chloroform extraction and ethanol precipitation. LINC00472 promoter in the samples was confirmed by qPCR using the primers: CTTTCCGACACCTGATT (SEQ ID NO. 1) (forward) and TAGCCAATTGGGGTCTTTG (SEQ ID NO. 2) (reverse).

### TNF-α Treatment

TNF-α (Sigma-Aldrich), diluted in the culture medium immediately before experiment, was added to cultured cells with a final concentration of 10 ng/ml. The cells were incubated for 24 hours before analysis. DMSO (Sigma-Aldrich) treated cells were used as control. Total RNA and proteins were extracted for analysis of LINC00472 expression and NF-κB activation.

### Tamoxifen Treatment

T-47D were cultured in the complete culture medium supplemented with 10 μM 4-Hydroxytamoxifen (Sigma-Aldrich), and the medium was changed every two days. After eight days of culture, cells were collected, and total RNA and proteins were extracted for qRT-PCR analysis of LINC00472 and western blot of ERα and NF-κB, respectively.

### Analysis of Cell Metabolomics

Cell lysates were prepared following protocols described previously (16-18). Briefly, appropriate weight of homogenizer beads and 50 μl of cold water were added to cell samples for initial extraction. A 270 μl mixture of ethanol and chloroform, 3:1 (v/v), was added to the initial extracts for second extraction. The final extracts were centrifuged at 14,500 rpm for 20 min at 4° C. The supernatants were used for targeted metabolic profiling of 140 lipids with an Acquity ultra-performance liquid-chromatography coupled with a Xevo TQ-S mass spectrometry (UPLCTQ-MS, Waters Corp.) The same materials were also utilized for untargeted metabolic profiling using an Agilent 7890A gas chromatography with a Leco Pegasus time-offlight mass spectrometer (GC-TOF-MS, Leco Corp). The raw UPLCTQ-MS data files were processed with Target Lynx Application Manager (Waters Corp.) to extract peak area and retention time of each metabolite. The raw GCTOF-MS data files were processed with Chroma TOF software (v4.22, Leco Corp.) which performed de-noising, peak detection and compound deconvolution. Internal standards and any known artificial peaks, such as peaks caused by noise, column bleed and BSTFA derivatization procedure, were removed from the data set. For UPLCTQ-MS, metabolite annotation was performed by comparing the accurate mass (m/z) and retention time (Rt) of reference standards in our in-house library and the accurate mass of compounds obtained from the web-based resources such as the Human Metabolome Database. For GC-TOF-MS, metabolites were identified by comparing the mass spectral similarity and retention index distance between the samples and standards of our inhouse library. A similarity score of more than 70% was selected for identification.

### Microarray Analysis of Gene Expression

Total RNA was extracted from cell lines using the method described earlier. The RNA quality was evaluated with absorbance and RNA Integrity Number (RIN) using the NanoDrop 2000 spectrophotometer (Thermo Fisher Scientific) and Agilent 2100 Bioanalyzer System (Agilent Technologies), respectively. Gene expression data were generated using the Affymetrix Human Transcriptome Array 2.0 (Affymetrix). DNA labeling, probe hybridization, and signal scanning were performed by the Genomic Shared Resource at University of Hawaii Cancer Center.

Expression intensities were stored as CEL-files which were processed using the robust multiarray average (RMA) algorithm in the Affymetrix Expression Console for inter-chip quantile normalization. Transcriptome Analysis Console (TAC) v3.0 (Affymetrix) was used to identify genes which were differentially expressed between mock and LINC00472 cells.

### Bioinformatics and Statistical Analysis

Ingenuity Pathway Analysis (IPA) was used to analyze the transcriptomic and metabolomic data in prediction of the biological network associated with LINC00472 overexpression. Downstream pathways and upstream signals suggested by IPA in both cell lines were considered as positive leads for further evaluation of their relationship with LINC00472 in cell experiments. PROMO was employed with 5% dissimilarity for predicting the binding sites of transcription factors in the LINC00472 promoter.

Values of LINC00472 expression in our study and from online datasets were log 2 transformed, and their differences by ER status were compared using the Student t test. Meta-analysis was performed to evaluate LINC00472 expression in association with ER status after the expression was dichotomized using the study-specific median as cutoff. For survival analysis, LINC00472 expression was also dichotomized as described above. Unadjusted hazards ratios (HRs) and 95% confidence interval (CI) for relapse and death were calculated for each dataset. Pooled HR and 95% Cl were estimated using the random-effect model (the DerSimonian and Laird method). Comprehensive Meta-Analysis (v2.0, BIOSTAT) was used for meta-analysis. Values shown in cell experiments were means and standard deviations (SD). Two-tailed Student t-test was used to compare means between groups. Paired t-test was used to compare paired differences in the same animals. Two-side p value less than 0.05 was considered statistical significance. Statistical Analysis System v9.4 (SAS Institute Inc.) was used for data analysis.

### Results

### LINC00472 Expression and Breast Cancer Survival

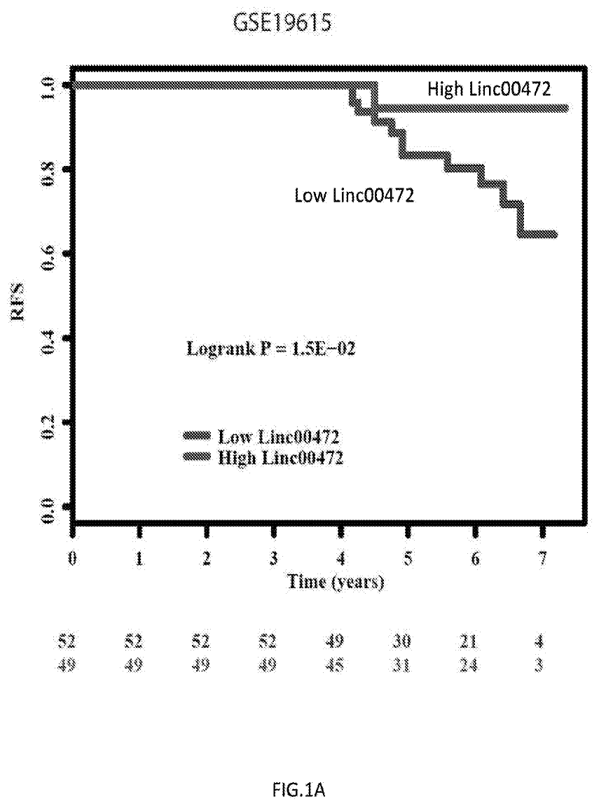

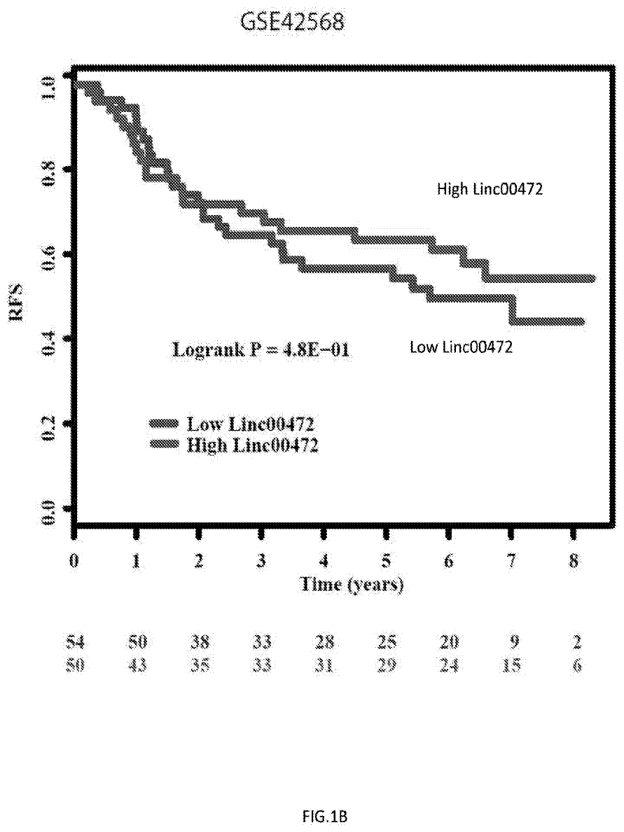

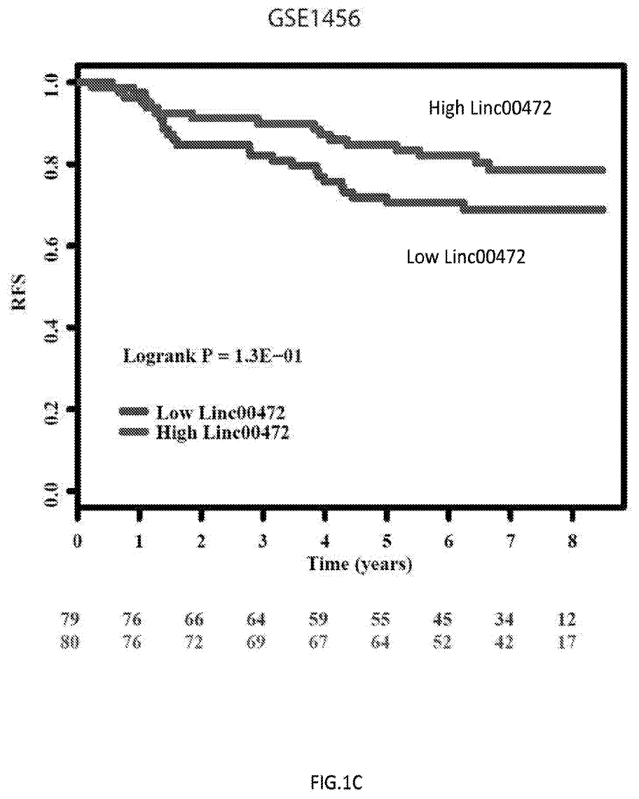

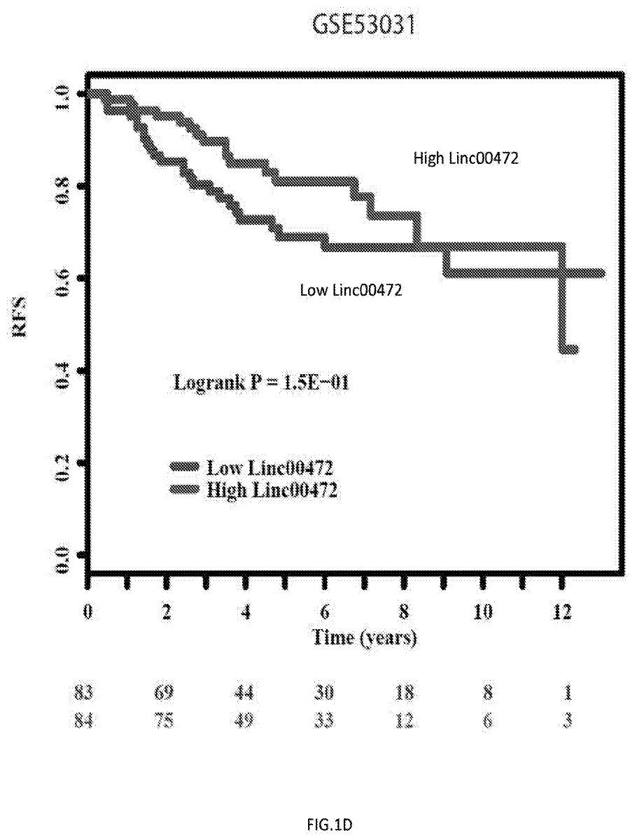

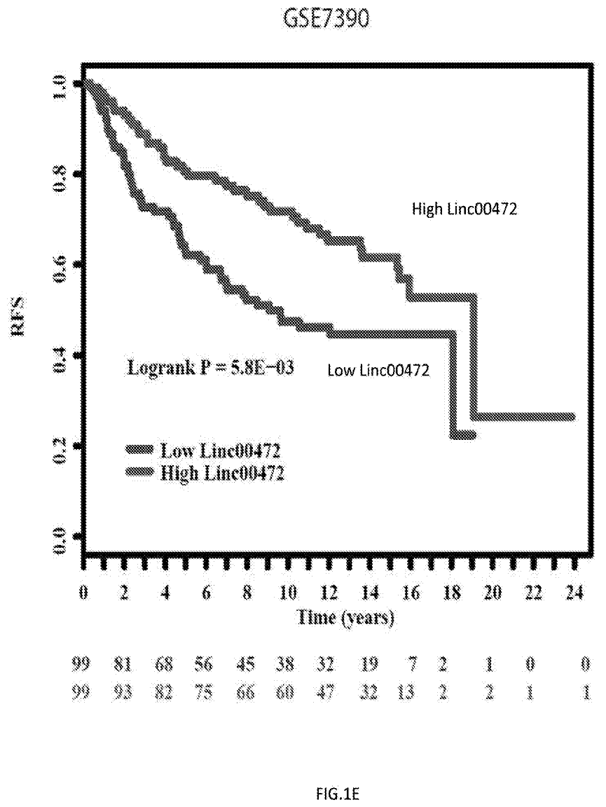

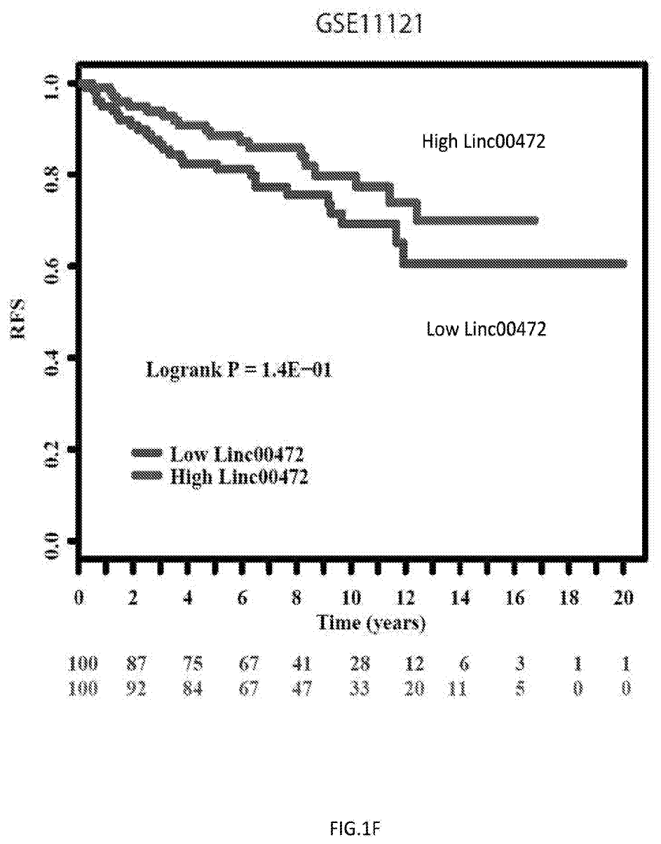

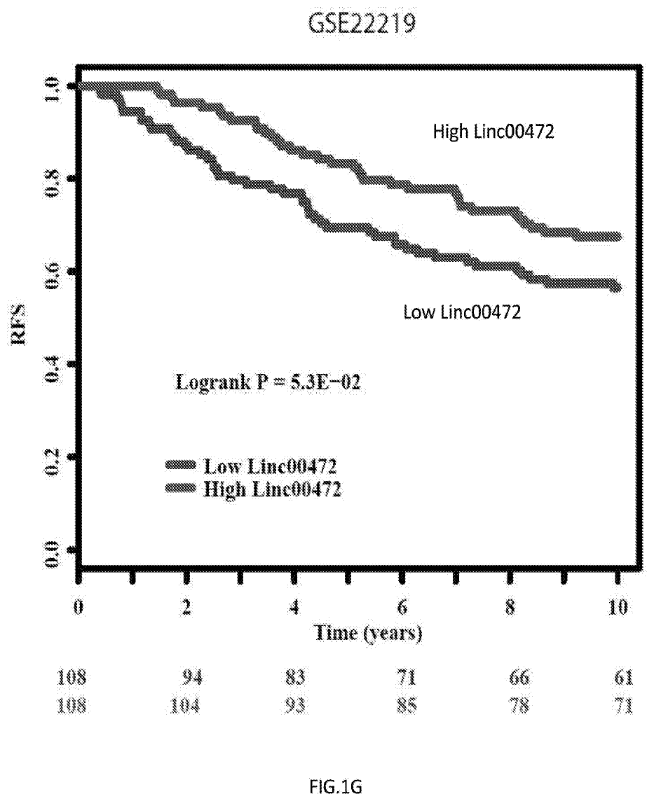

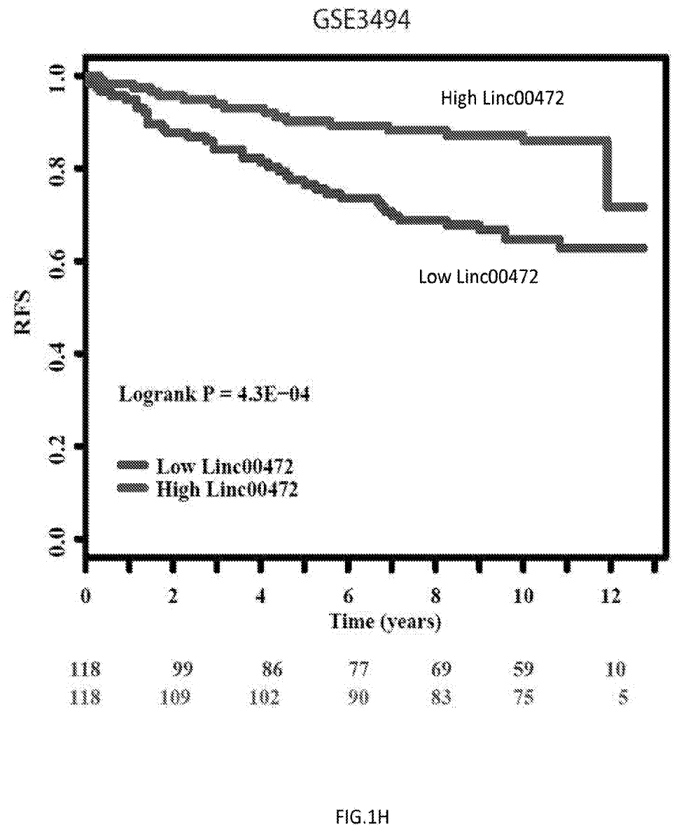

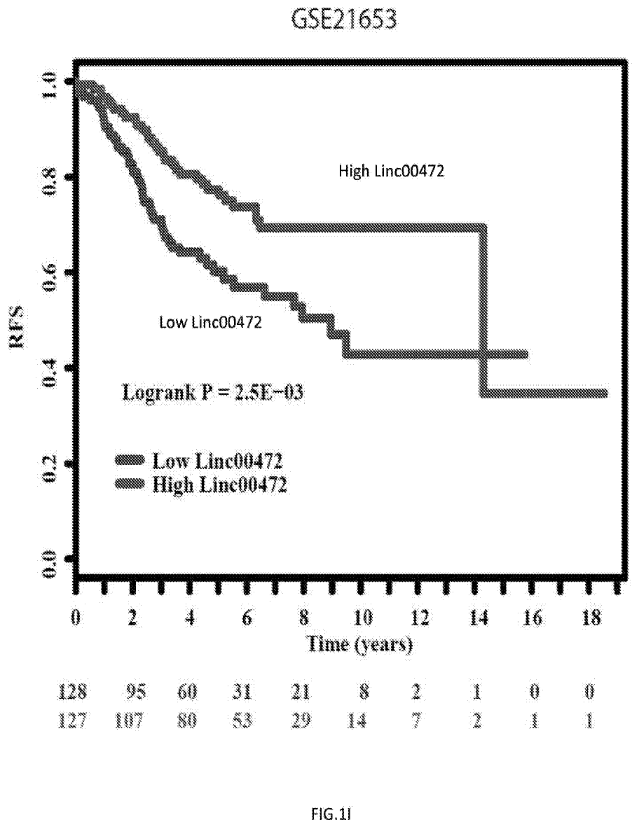

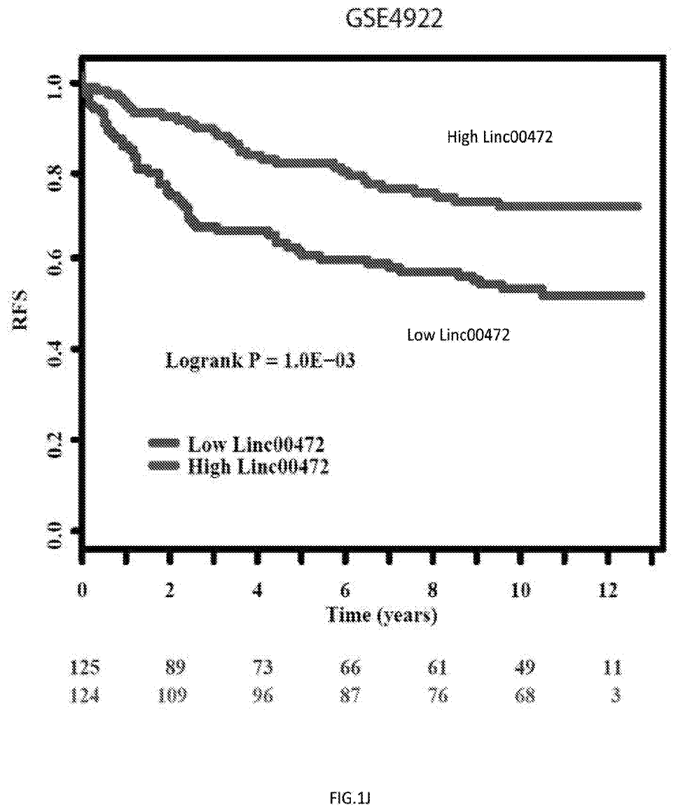

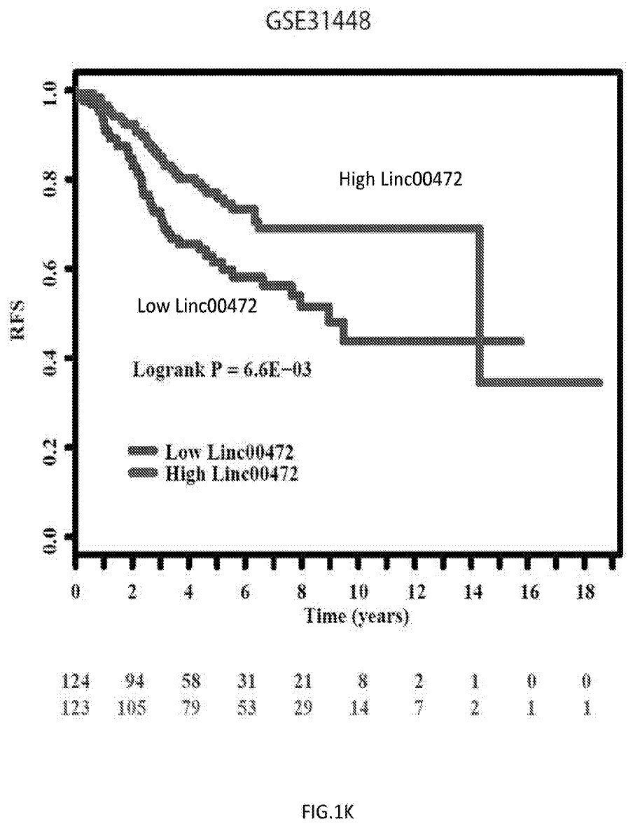

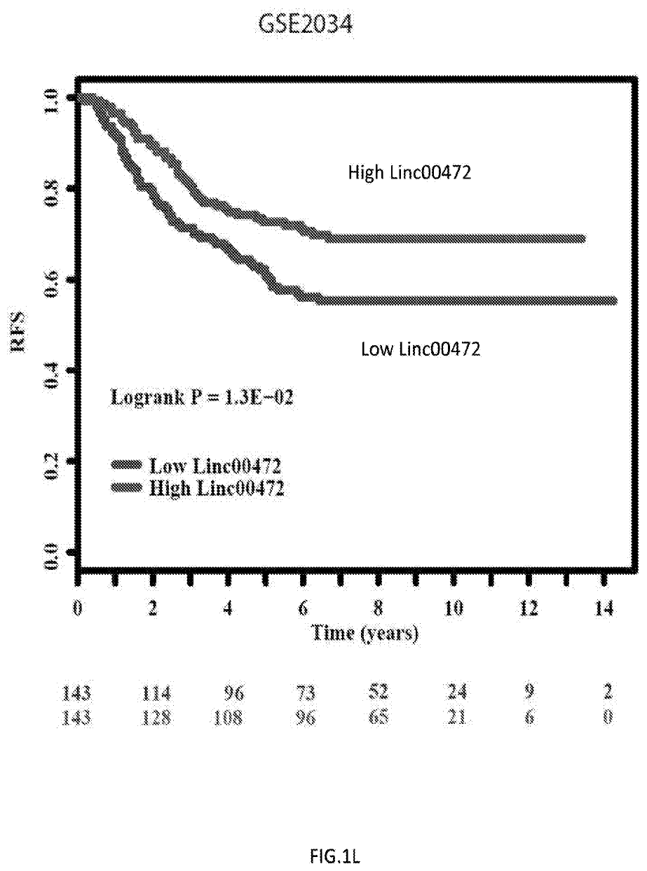

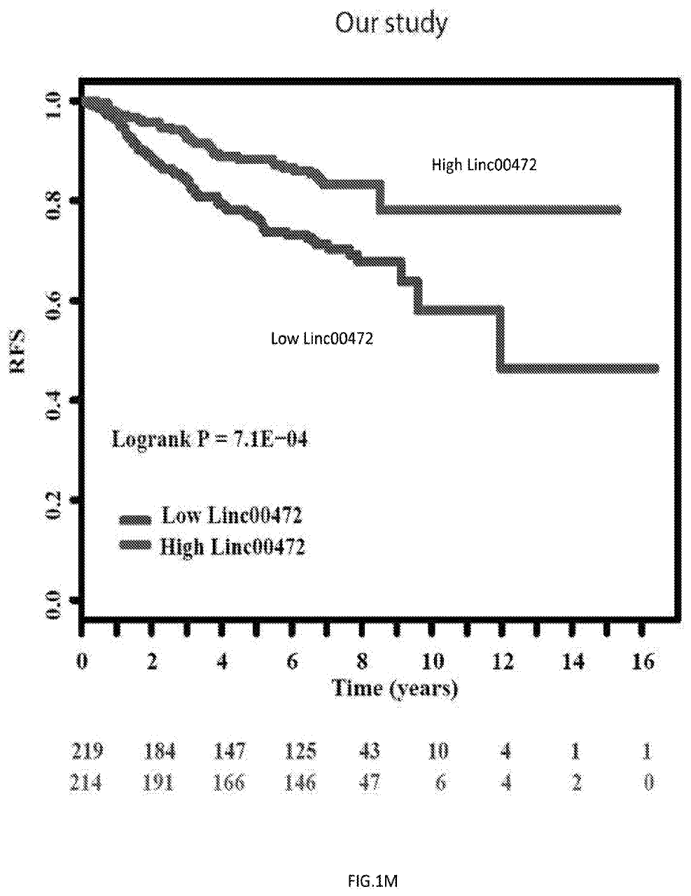

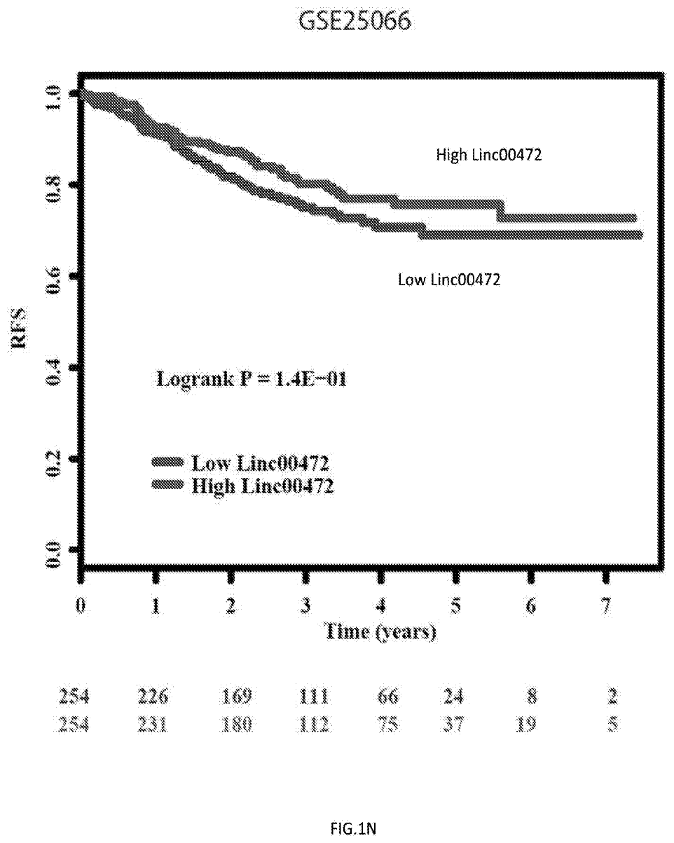

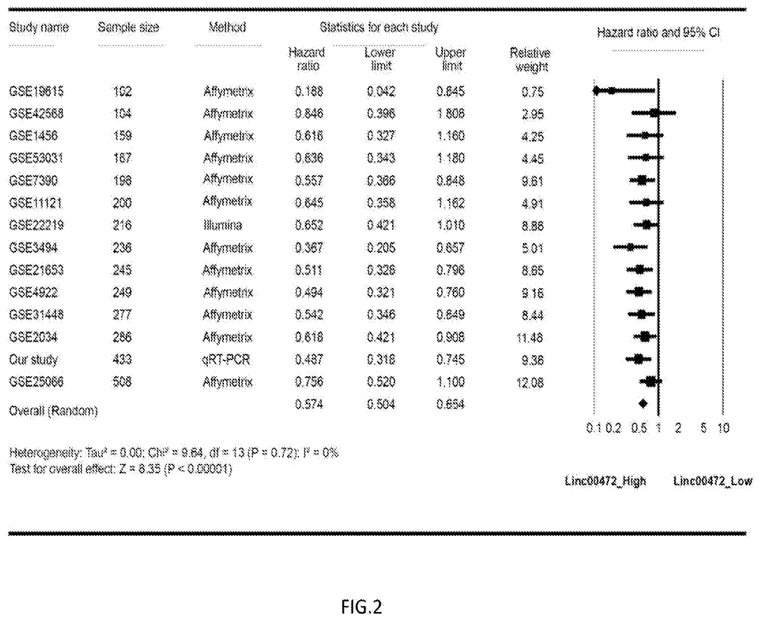

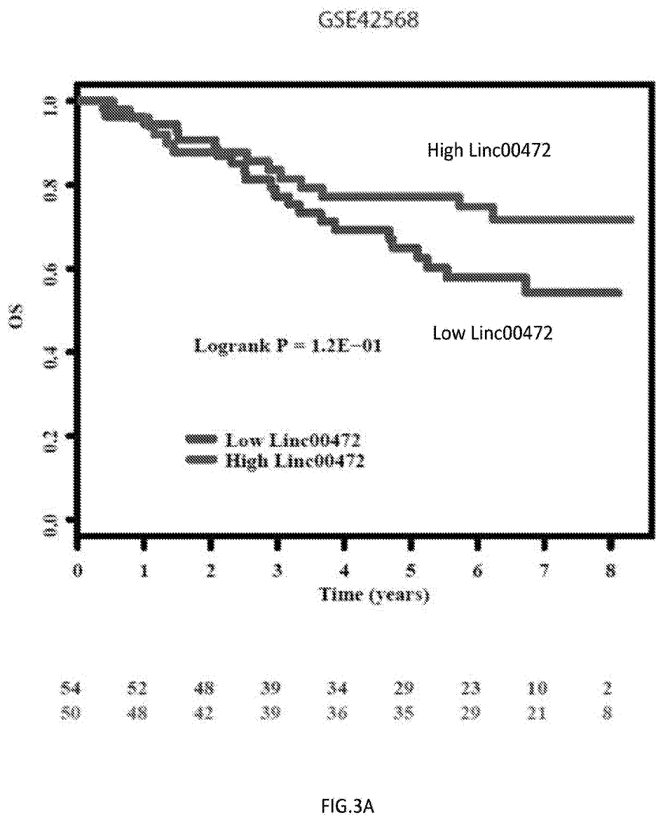

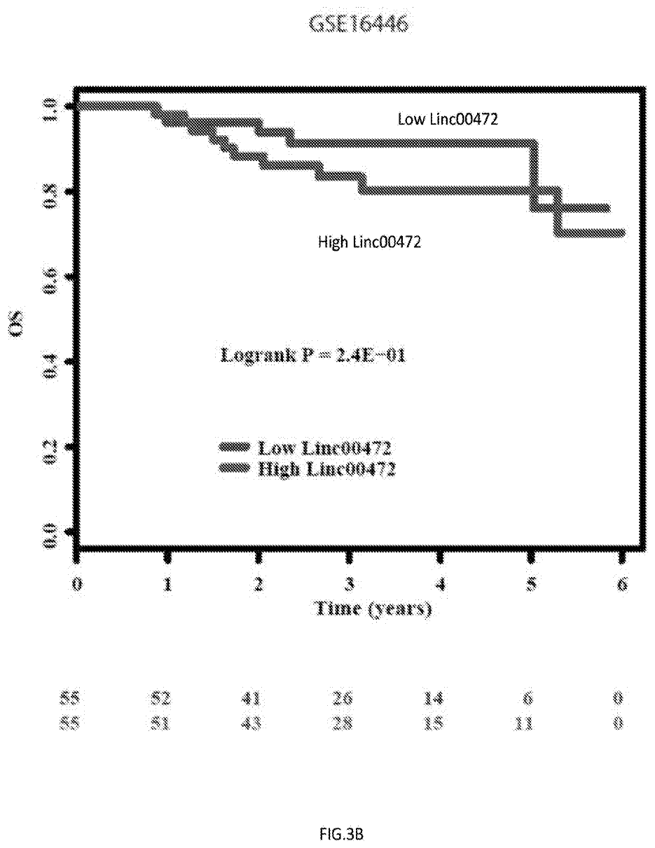

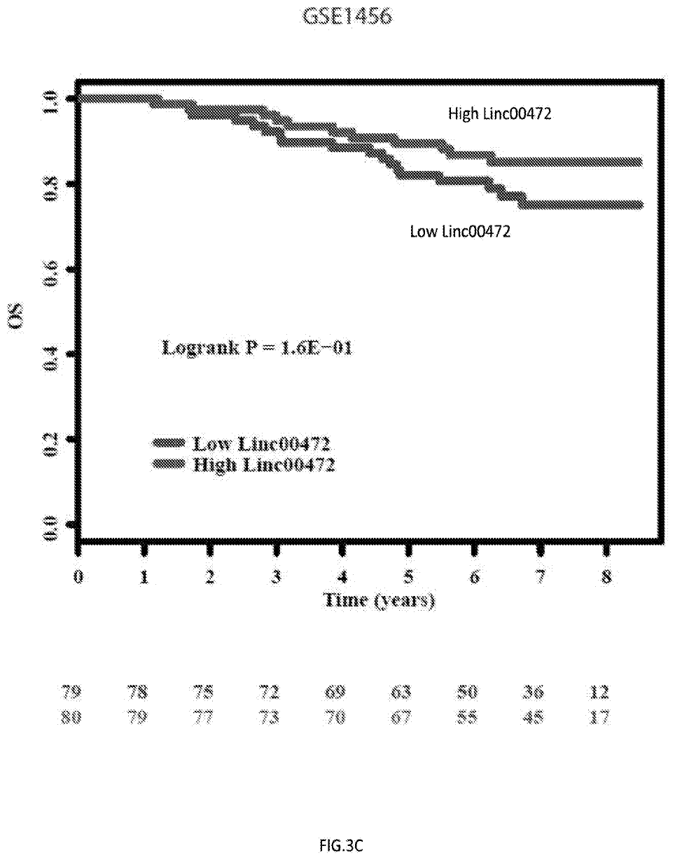

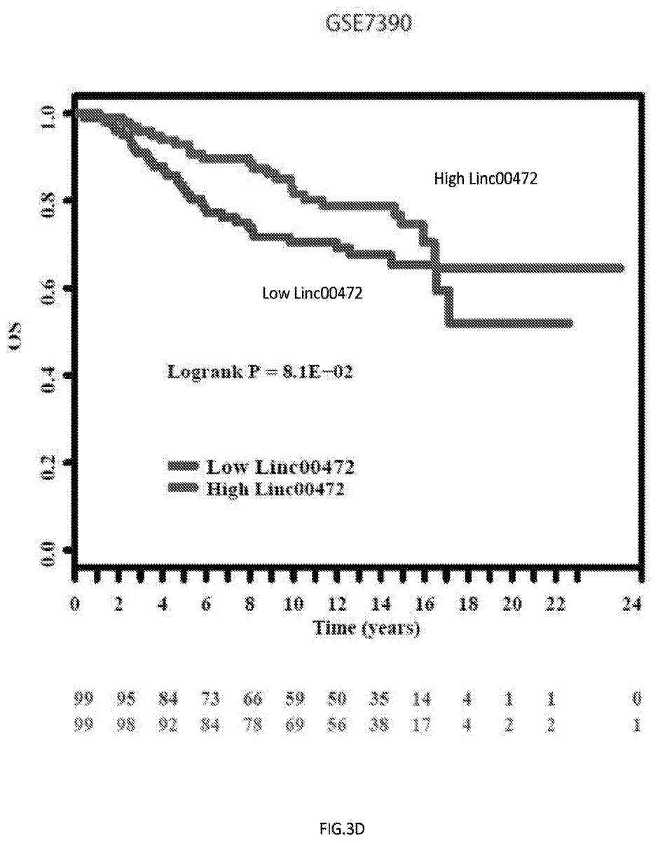

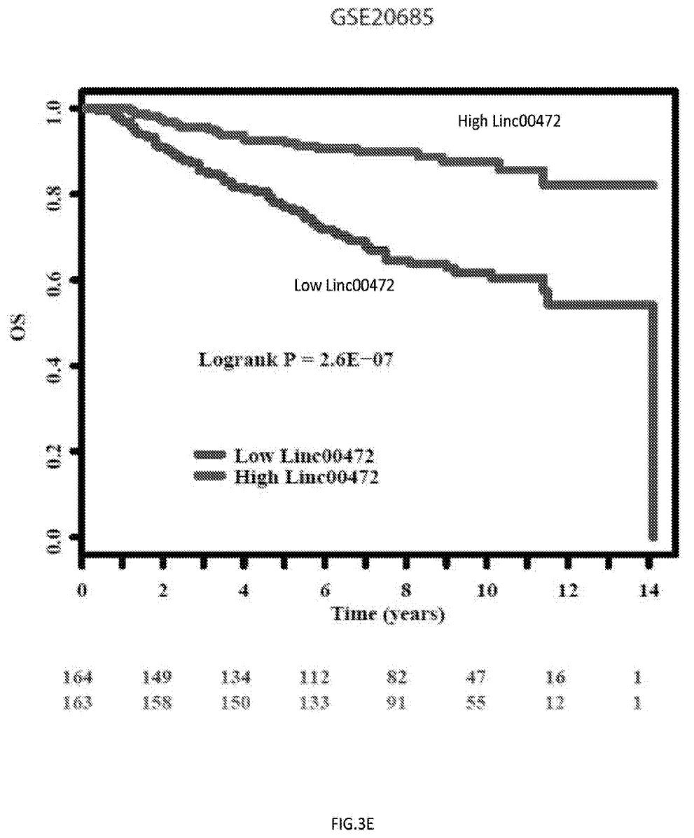

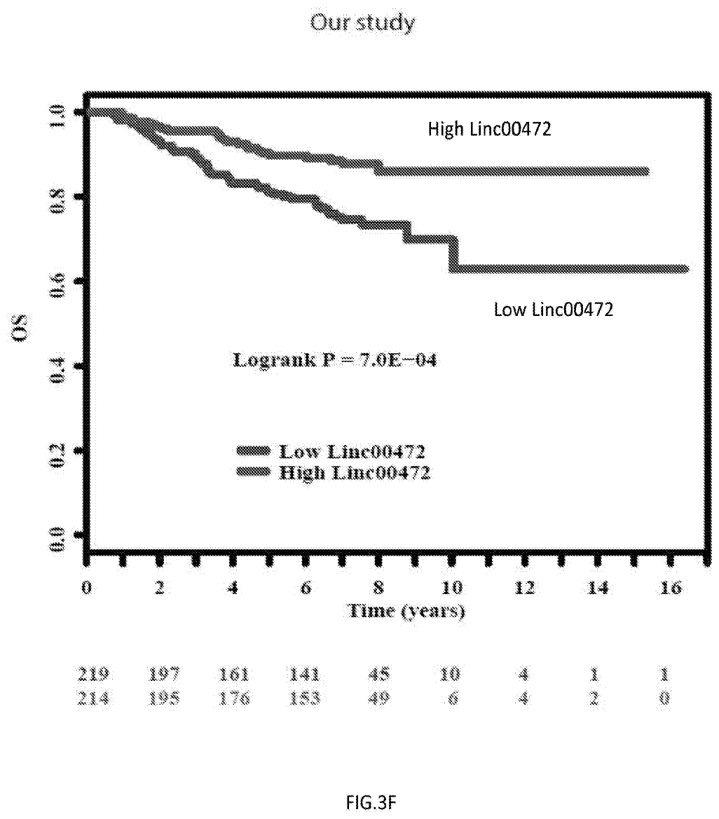

FIGS. 1A-1N, 2, 3A-3F, and 4 illustrate the association of LINC00472 expression with breast cancer survival. As shown in FIGS. 1A-1N, a meta-analysis of our updated study and the 13 GEO datasets newly extracted from the NCBI database showed that high LINC00472 expression in breast cancer was associated with longer relapse-free survival compared to low expression. As shown in FIG. 2, the summarized hazard ratio (HR) among all the studies was 0.574, and 95% confidence interval (CI) ranged from 0.504 to 0.654. As shown in FIG. 3A-3F, a meta-analysis also revealed that high expression of LINC00472 was associated with favorable overall survival. Furthermore, as shown in FIG. 4, Patients with high LINC00472 had more than 40% reduction in risk of death, compared to those with low LINC00472.

### LINC00472 Expression in Breast Cancer Cells

FIGS. 5A-5B, 6A-6D, 7A-7D, 8A-8D, 9A-9B, and 10 illustrate LINC00472 suppression of tumor cell proliferation, migration, invasion, and colony formation. As shown in FIG. 5A, LINC00472 expression was analyzed in 6 breast cancer cell lines, including three sex hormone receptor-positive (MCF-7, T47D, ZR-75-1), two triple-negative (MB231, Hs578T), and one Her2-positive (SKBR3), and the expression was low in all the cell lines, except T47D. To evaluate the effects of LINC00472 overexpression on aggressive breast cancer cells, MB231 and Hs578T were transfected with a L/NC00472-expressing plasmid. After transfection, as shown in FIG. 5B, LNC00472 expression was significantly increased in the cell lines, both in pool and a single clone.

### Tumor Suppression by LINC00472 In Vitro and In Vivo

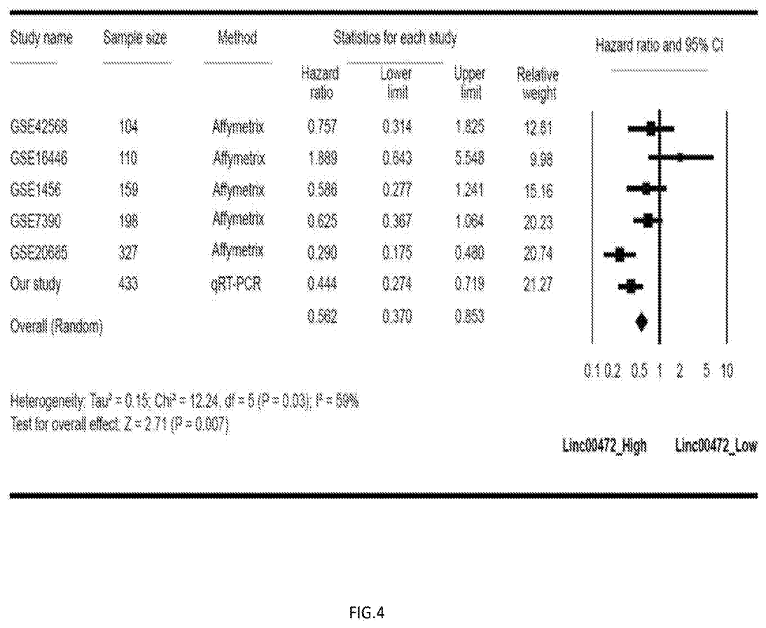

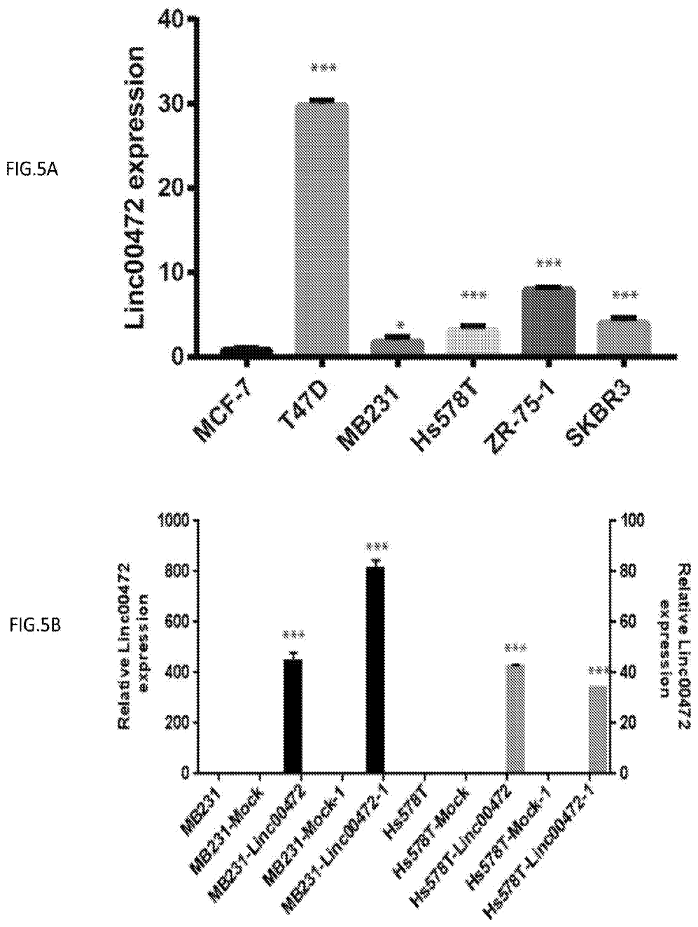

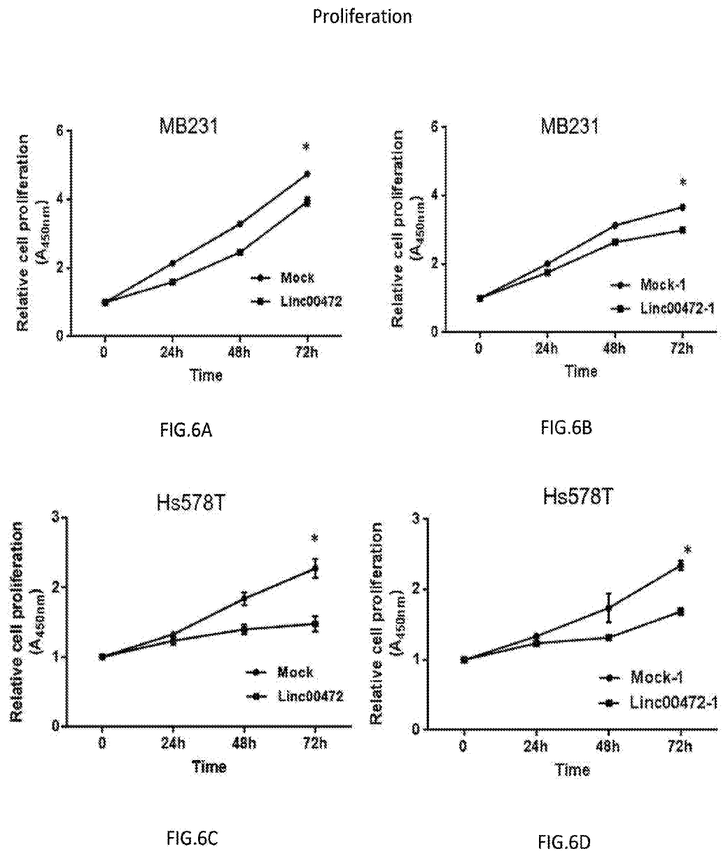

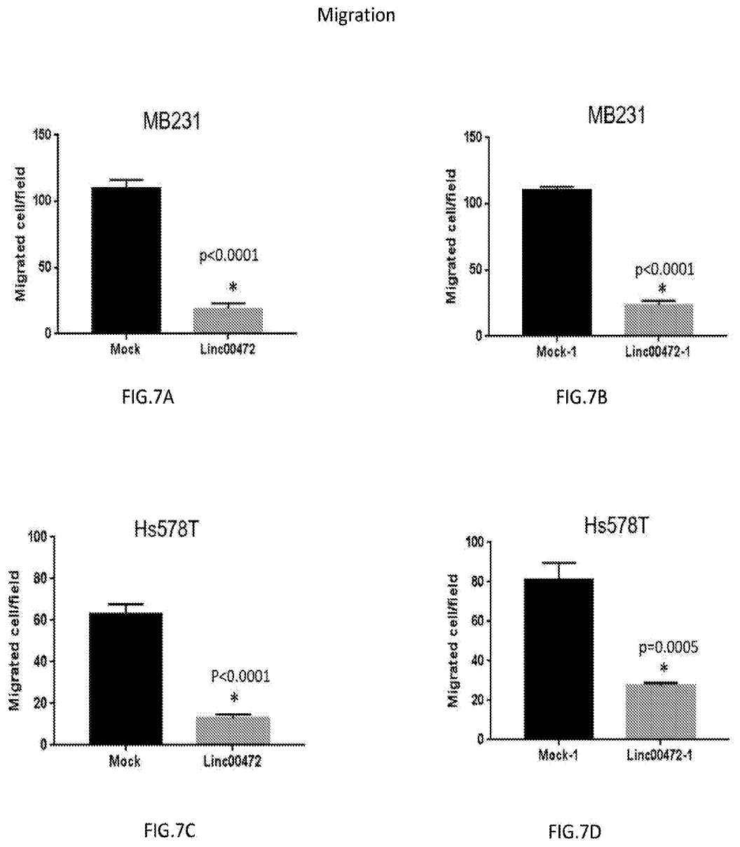

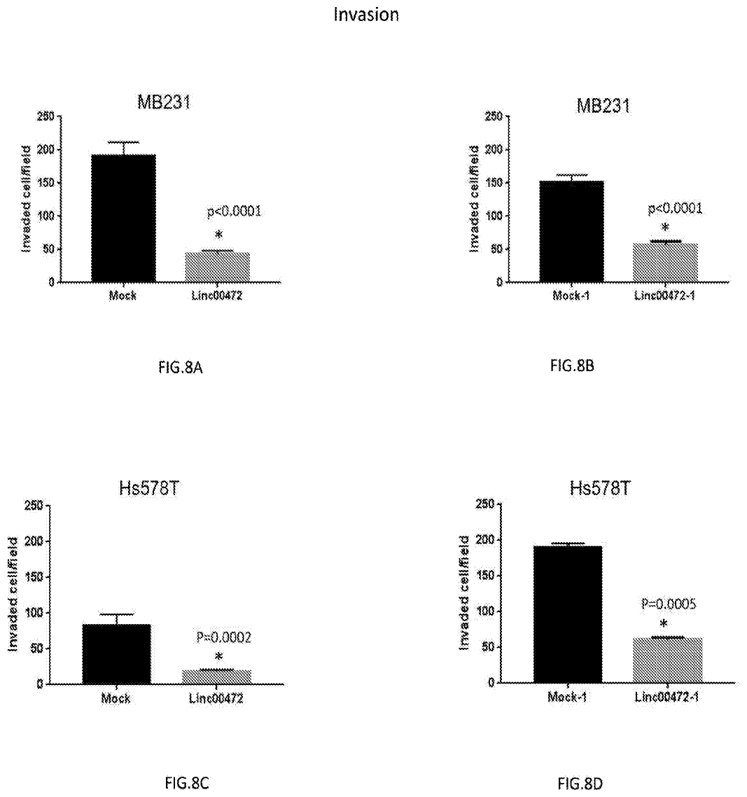

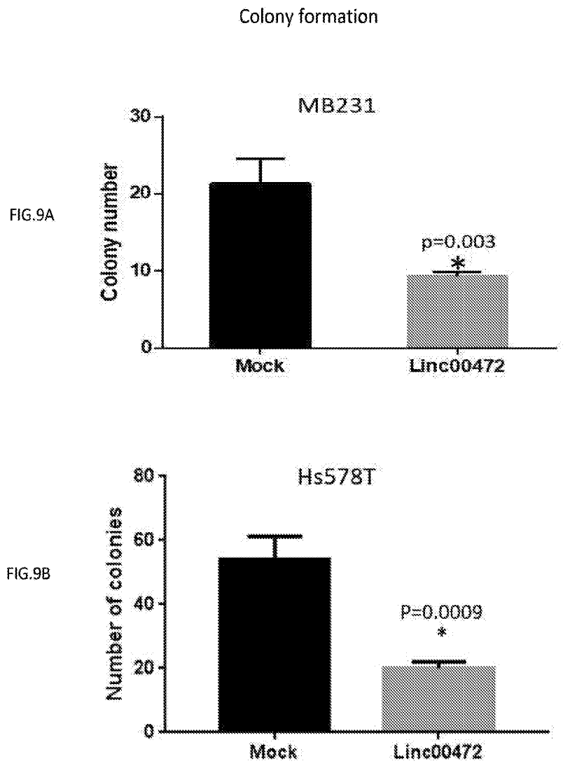

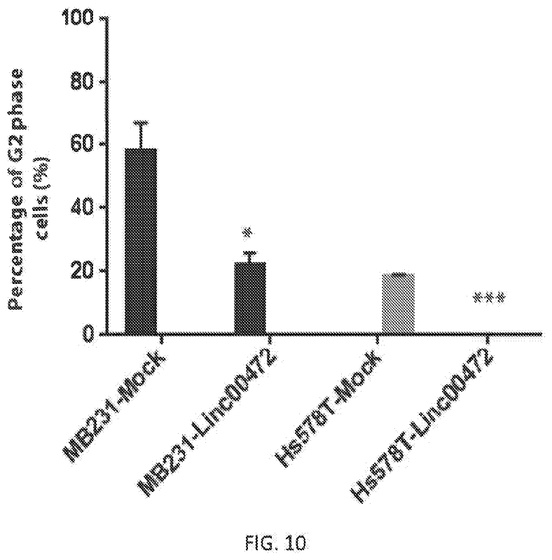

As shown in FIG. 6A-6D, LINC00472 overexpression significantly reduced cell proliferation. Moreover, as shown in FIGS. 7A-7D and 8A-8D, respectively, LINC00472 overexpression significantly inhibited cell migration and invasion. The overexpression cells were less likely to form colonies in soft agar (as shown in FIGS. 9A-9B), and had fewer cells in G2 phase (as shown in FIG. 10). All the findings were consistent between the two cell lines.

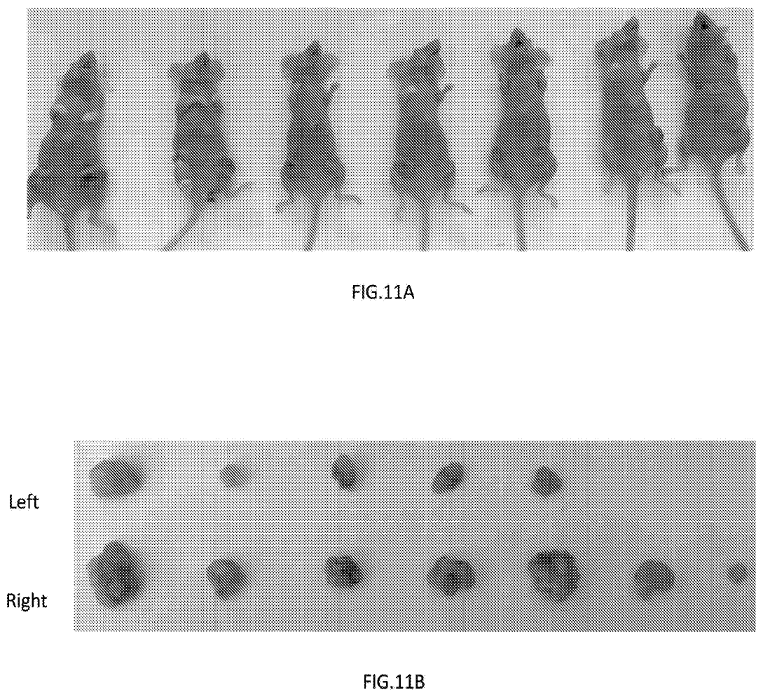

To examine the effect of LINC00472 in vivo, a xenograft model was developed by injecting MB231 cells into the BALB/c female nude mice. Using self-control, the tumor cells were implanted into the mammary fat pad, LINC00472 overexpression cells on the left and mock cells on the right. FIGS. 11A-11E show LINC00472 inhibition of breast tumor growth in the xenograft mouse model. More specifically, tumors from the LINC00472 overexpression cells (left) grew much smaller (as shown in FIGS. 11A and 11B), with slower tumor growth rate (as shown in FIG. 11C) and higher LINC00472 expression in the tumors (as shown in FIG. 11D). Tissue analysis showed less malignant morphology in tumors with LINC00472 overexpression (left) than in those without overexpression (as shown in FIG. 11E).

### LINC00472-Related Transcriptomes and Metabolomes

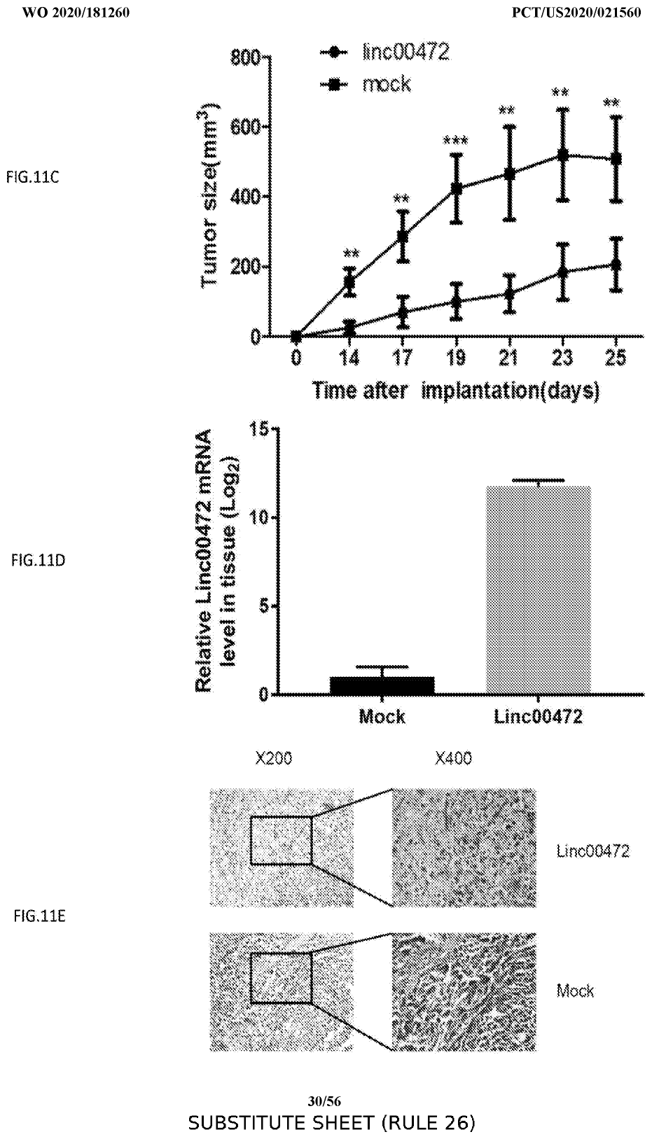

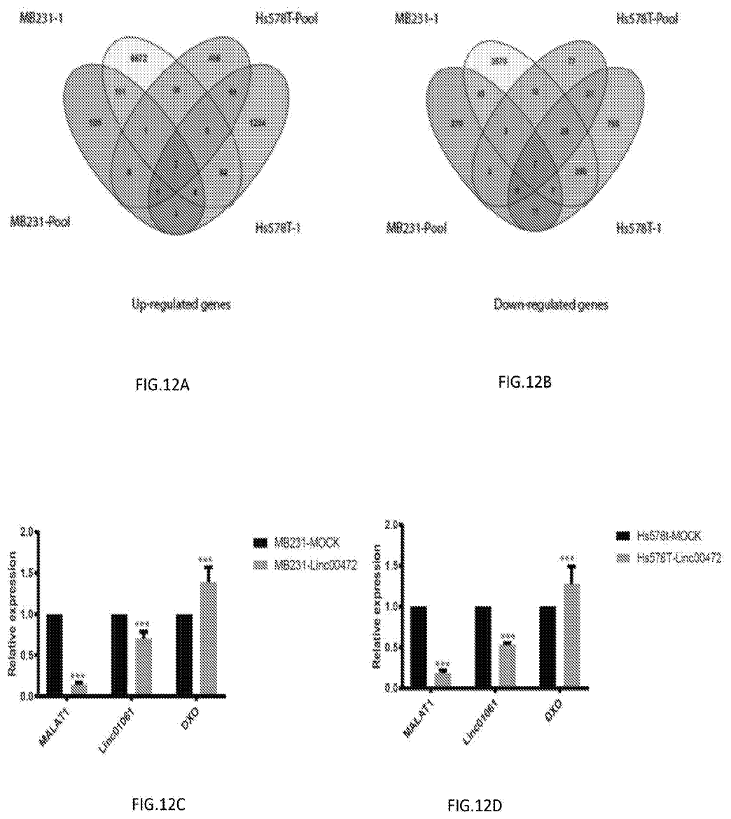

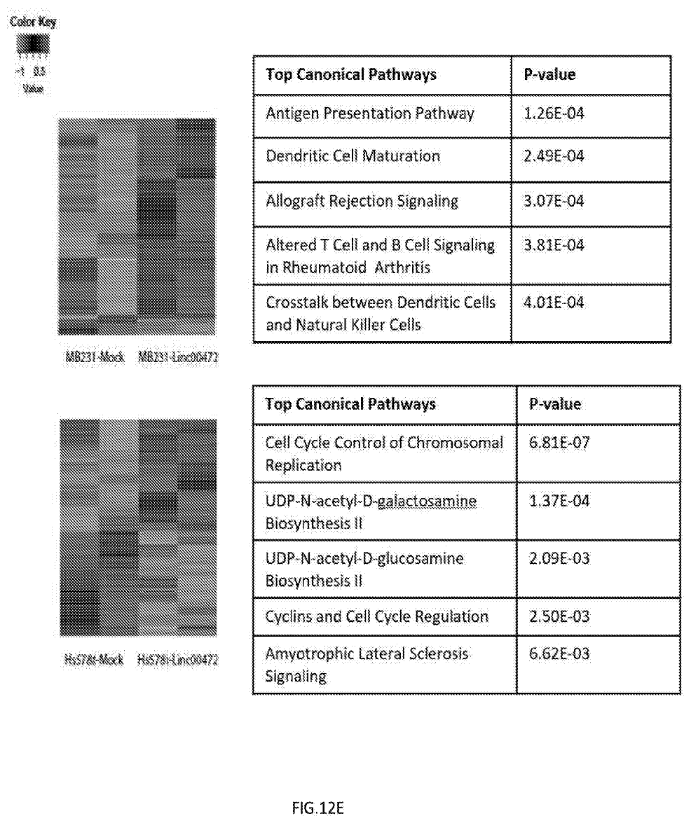

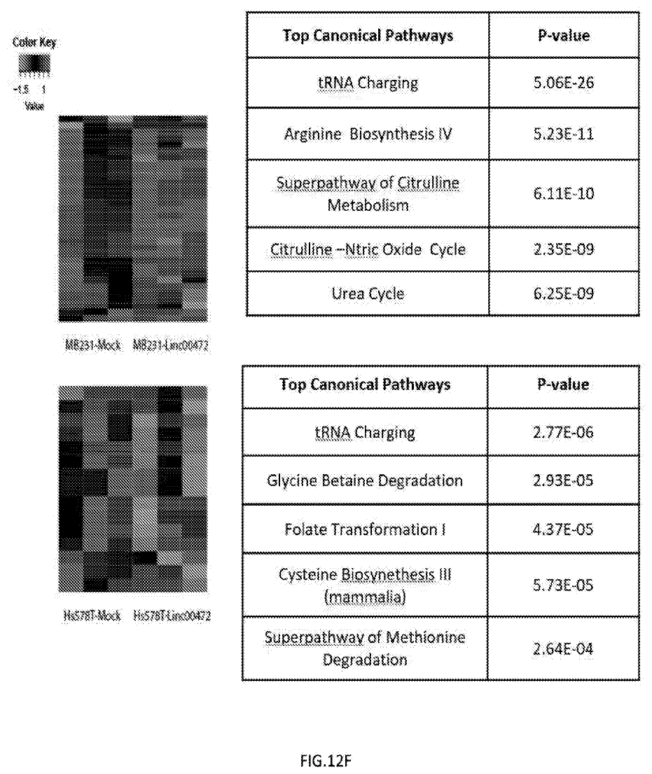

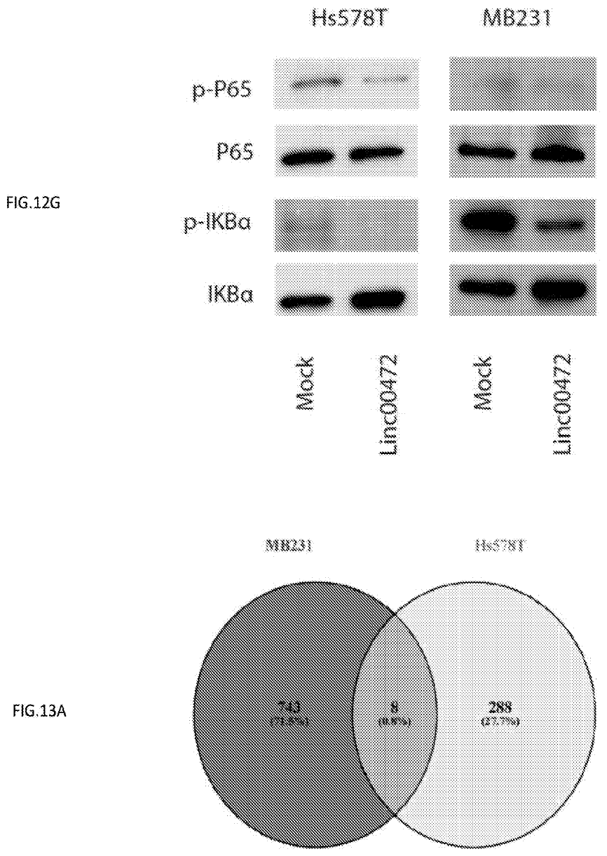

Gene expression profiles of MB231 and Hs578T cells were analyzed with an Affymetrix microarray chip. FIGS. 12A-12G further illustrate analysis of transcriptome, metabolome and NF-κB inactivation associated with LINC00472 overexpression. Comparing the expression data from 8 cell samples (two pools and two single colons in each of the two cell lines), it was found that 2 up- and 2 down-regulated transcripts were shared by all the samples (as shown in FIGS. 12A-12B), including LINC00472, DXO, LINC01061 and MALAT1. Upregulated LINC00472 expression was expected since the cells were transfected with a LINC00472 plasmid. To validate the microarray results, qRT-PCR was performed on three top transcripts, and the findings were consistent (as shown in FIGS. 12C-12D). The expression profiles were interrogated for biological network using the Ingenuity Pathway Analysis (IPA). FIG. 12E shows the top five canonical pathways where significant gene enrichment was observed due to LINC00472 overexpression. The two cell lines had different top network, but downregulation of TNF-111 signaling was indicated by IPA in both cell lines.

In metabolomics analysis, 30 metabolites were found in MB231 and 15 in Hs578T that were significantly associated with LINC00472 overexpression. IPA analysis of these metabolites showed that the top five canonical pathways were different between the cell lines, except for tRNA Charging (as shown in FIG. 12F). Upregulation of superpathway of methionine degradation was indicated by IPA in both cell lines.

### NF-κB Suppression by LINC00472

Following the IPA results, it was tested if TNF-α could affect LINC00472 expression in breast cancer cells. Our experiments showed no effect of TNF-α on the lncRNA, but TNF-α activation of NF-κB, a downstream target of TNF-α was suppressed by LINC00472 overexpression.

Based on the observation and possible involvement of NF-κB in the superpathway of methionine degradation suggested by our metabolomics analysis, the impact of LINC00472 on NF-κB was examined, and significant decreases in phosphorylation of IκBα and p65 was found when LINC00472 was overexpressed (as shown in FIG. 12G), suggesting NF-κB being a possible target of LINC00472.

### Relationship of ERα, LINC00472 and NF-κB

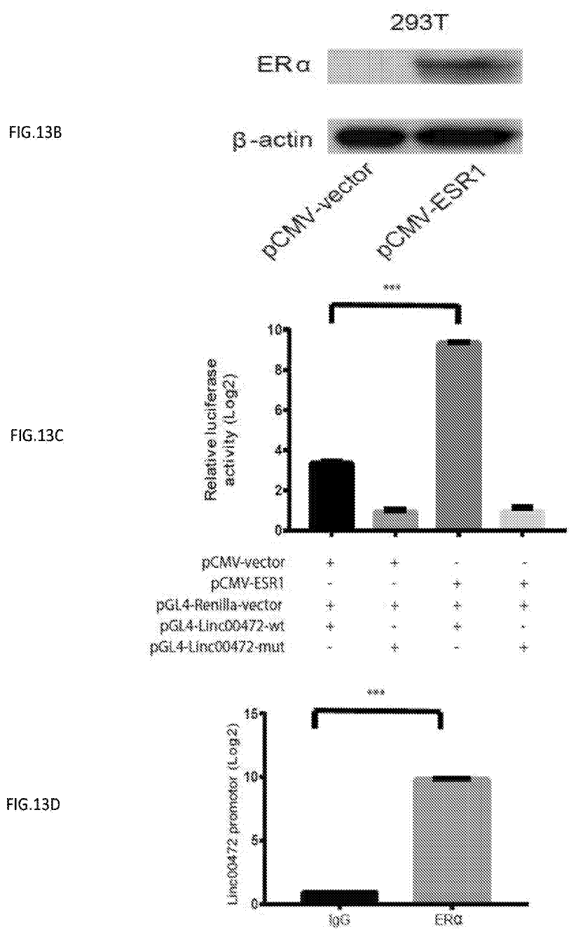

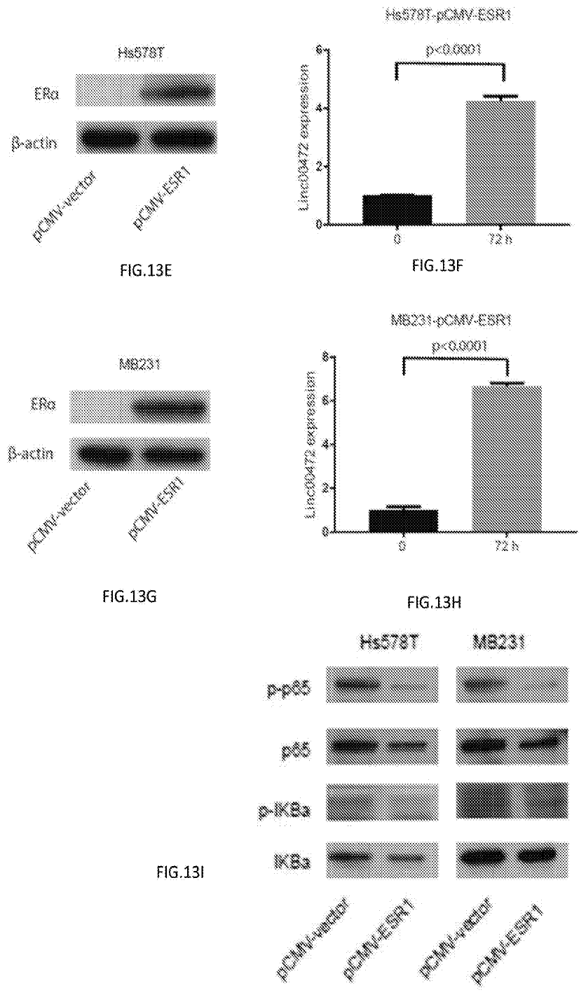

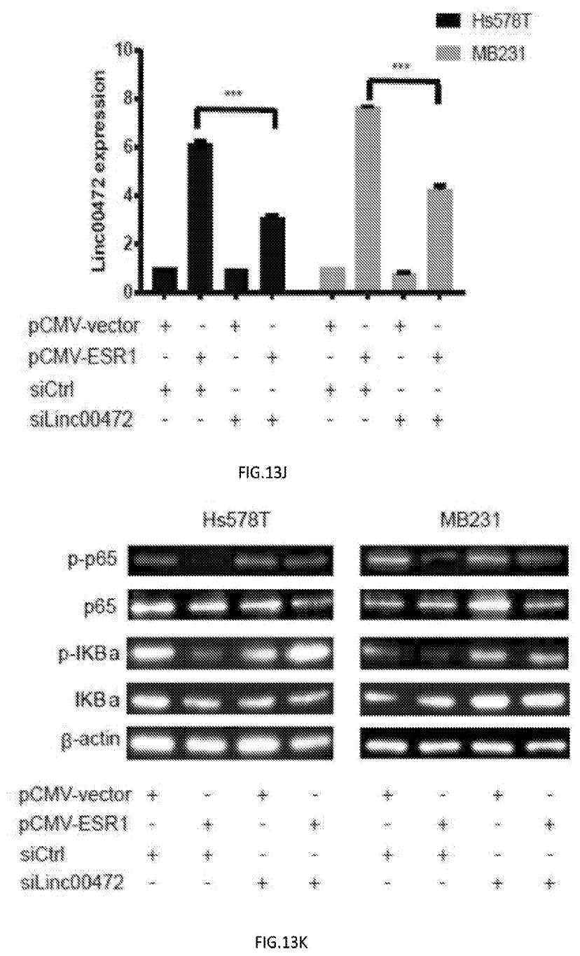

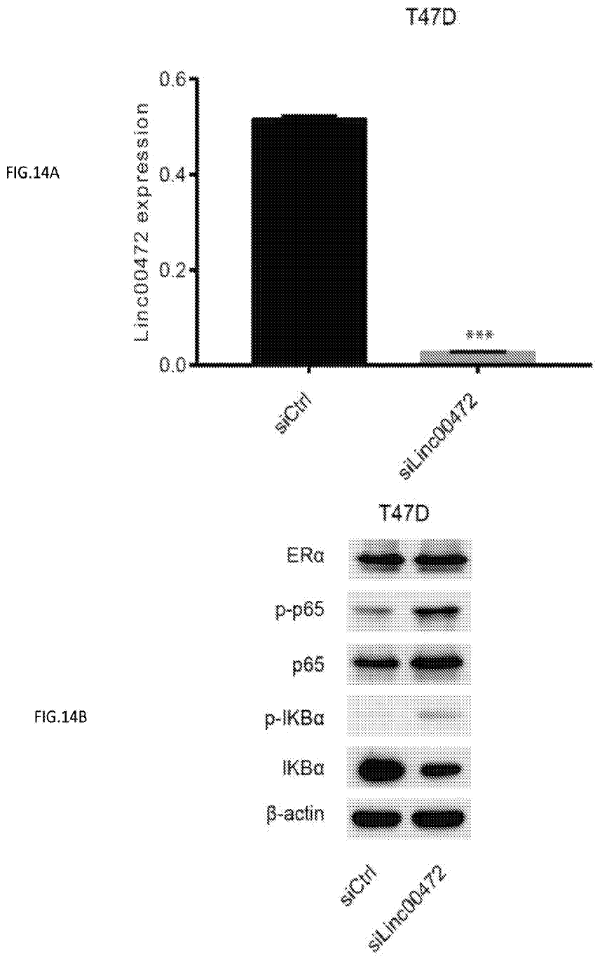

Using IPA, the expression profiles were also interrogated in prediction of signals involved in the regulation of LINC00472 expression. Eight molecules were suggested as possible signals shared by both cell lines (as shown in FIG. 13A), one of which was ESR1, the estrogen receptor alpha (ERα) gene. In search for transcription factors for LINC0047, the promoter sequence of LINC00472 (SEQ ID NO: 3, as shown in the table below) was interrogated using the PROMO. The software identified a region, −591 to −595, as a possible binding site for ERα.

Linc00472 promotor sequence (SEQ ID NO: 3)

| | |
|---|---|
| 1 | ACATGGTGGTGCCAGTGACAGTCTGTGTTTTGGGGCAGGATAGAAACTAA |
| 51 | CCTGCTTCAGTTACCTGACATTCTAGCAAACTTATTAGGAACACAAACGC |
| 101 | TCGGGTCTCTGAATGCACCAACCTGAGCGCCGGCTGATTAGGCATTGTAA |
| 151 | AGCAGTGTTCTAAAAGGAAAGGCCTTCACTTAAACAGTGCTACAACGTGT |
| 201 | TTGTTCAGCTTTCTTTCATACACAAACTTTTGCCAGAAAAGGCGTTTTAA |
| 251 | GCCGAGGGTAAAGATTTTGTGCGTCCACCGTTCCCATCTTCAACTCTTTA |
| 301 | GAATAATAGTCATTTAAGCACCGGACCTGTCTTCAGATTCTTACTTTGCG |
| 351 | ACACAGCTTTGGCCGGACTTGGCTTGATCTGGGCTCCAGGATCGGTCCCA |
| 401 | CCACCCGGGCTCGGAGCGGTTTGTTCCTAGTGGATCAGGGCGGGTGTGTT |
| 451 | GCCGGAGTCGCCTTCTATTGGCTACACTCCCGGGGACTGGCTGGGCTTTC |
| 501 | CGACACCTGATTGGGCGGAACAGCCCTCTGTACGCCGACATCATTGGAGG |
| 551 | GCGCTGGAGCCAGGGGGCGGAGCGGGTTCCCCAGGATTCTTGACCGGGCG |
| 601 | CGCTAGTCCGTCCGCTGAGCCGGGCGCGGGGCGCAAGAGCGGAGCTGCGC |
| 651 | GAGCCGCTGCGGAGGGAAGGGCTCCTAGCCAATTGGGGTCTTTGAGGCGA |

A luciferase reporter assay was performed to test if ERα was able to interact with the LINC00472 promoter. The results showed that after overexpressing ESR1 in 293T (as shown in FIG. 13B), cells transfected with an intact promoter of LINC00472 had elevated luciferase signals, whereas the cells transfected with a mutant LINC00472 promoter that did not contain the ERα binding site had no increase in luciferase activity (as shown in FIG. 13C). A ChIP assay further demonstrated that ERα was able to bind to the LINC00472 promoter (as shown in FIG. 13D). Transfecting Hs578T and MB231 with an ESR1 plasmid (as shown in FIGS. 13E and 13G) increased LINC00472 expression in the cells (as shown in FIGS. 13F and 13H). Cells with overexpressed ERα had reduced phosphorylation of IκBα and p65 (as shown in FIG. 13I). Suppressing LINC00472 expression by siRNA (as shown in FIG. 13J) could abolish or reduce the suppression of NF-κB by ERα (as shown in FIG. 13K), suggesting that the inhibitory effect of ERα on NF-κB be mediated by LINC00472.

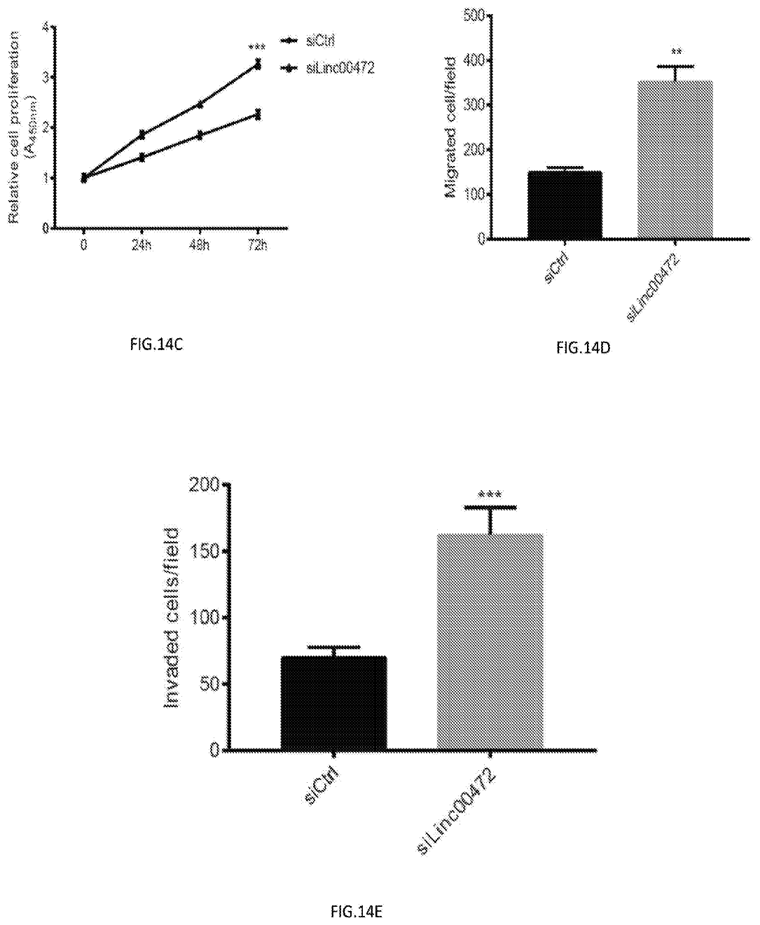

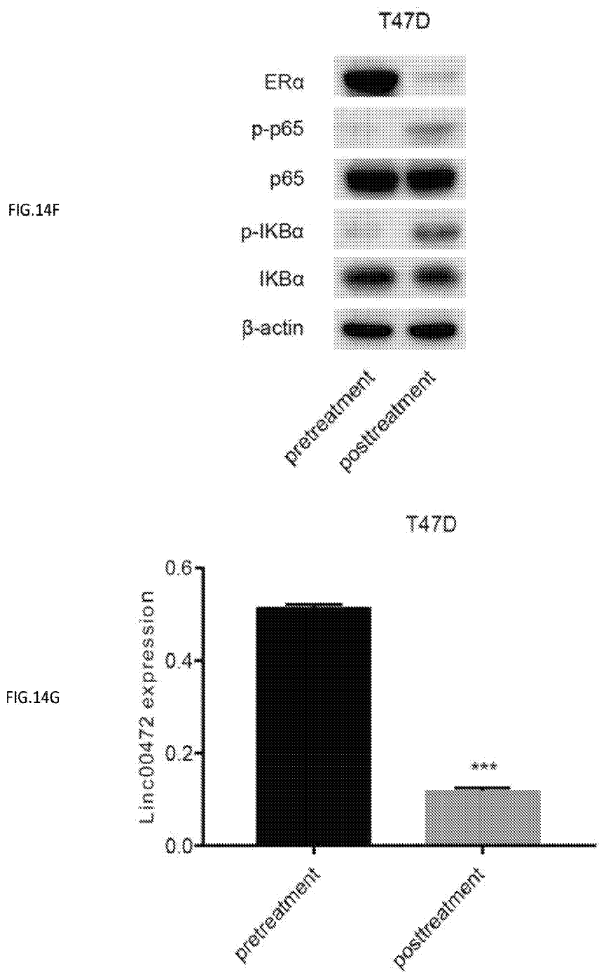

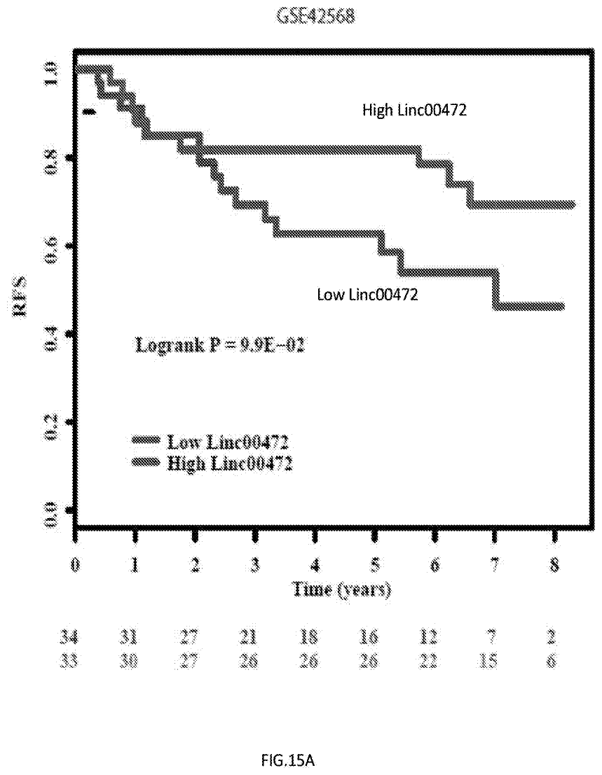

LINC00472-mediated ERα suppression on NF-κB was further verified in T47D cells after LINC00472 expression was suppressed by siRNA knockdown (as shown in FIG. 14A). Suppressing LINC00472 expression could increase the phosphorylation of IκBα and p65 while having no effect on ERα expression (as shown in FIG. 14B). Reducing LINC00472 expression by siRNA in T47D also increased cell proliferation (as shown in FIG. 14C), migration (as shown in FIG. 14D) and invasion (as shown in FIG. 14E). Also, T47D cells were treated with tamoxifen to examine the anti-ER effects on LINC00472 and NF-κB. As expected, the treatment lowered the expression of ERα and LINC00472, and increased the phosphorylation of IκBα and p65 (shown in FIGS. 14F and 14G), further confirming the relationships of ERα, LINC00472 and NF-κB.

Associations of LINC00472 Expression with ER Status and Patient Survival

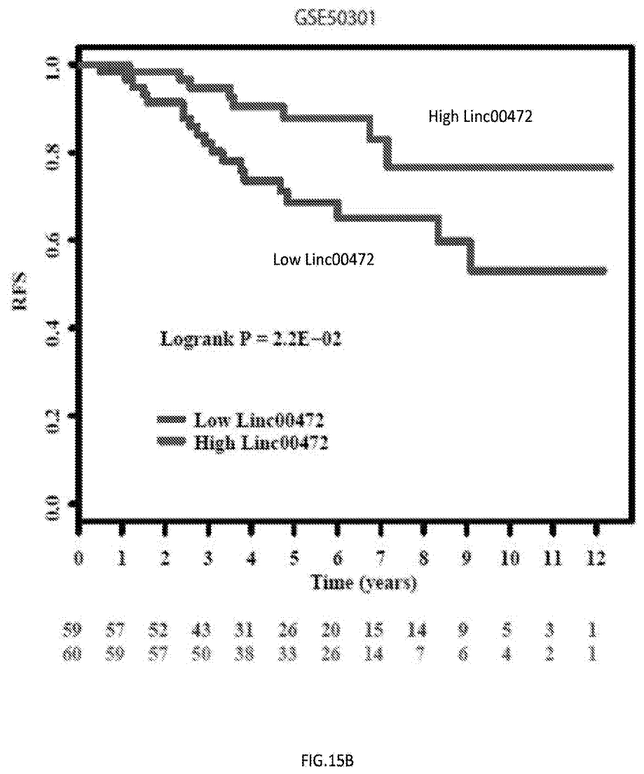

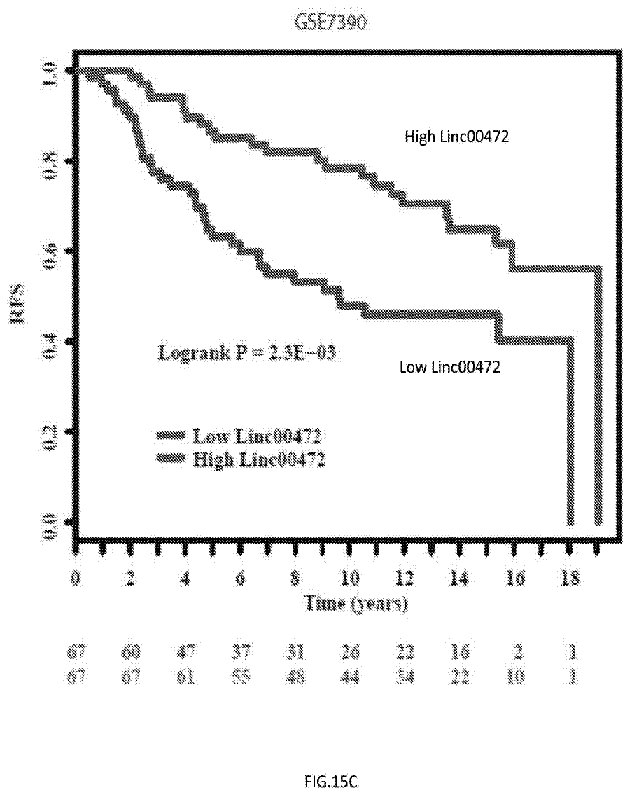

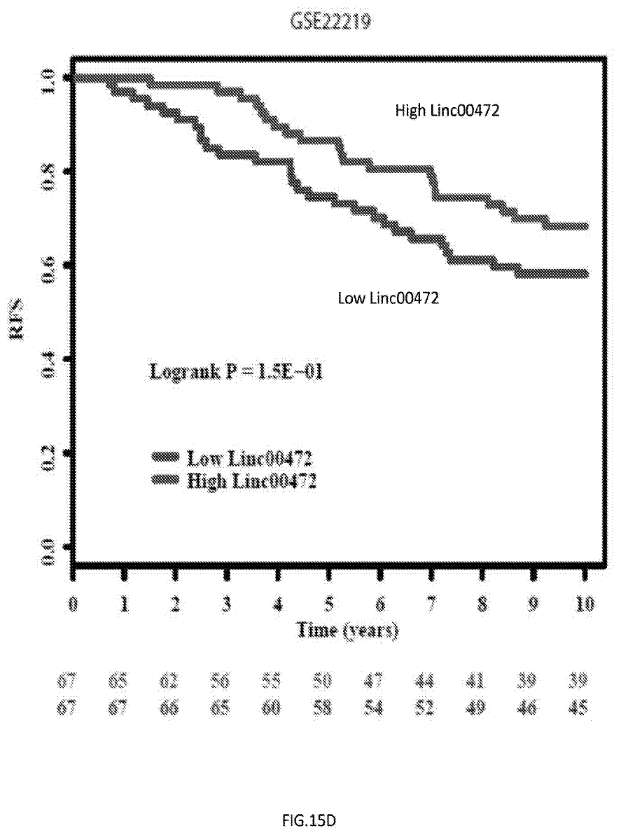

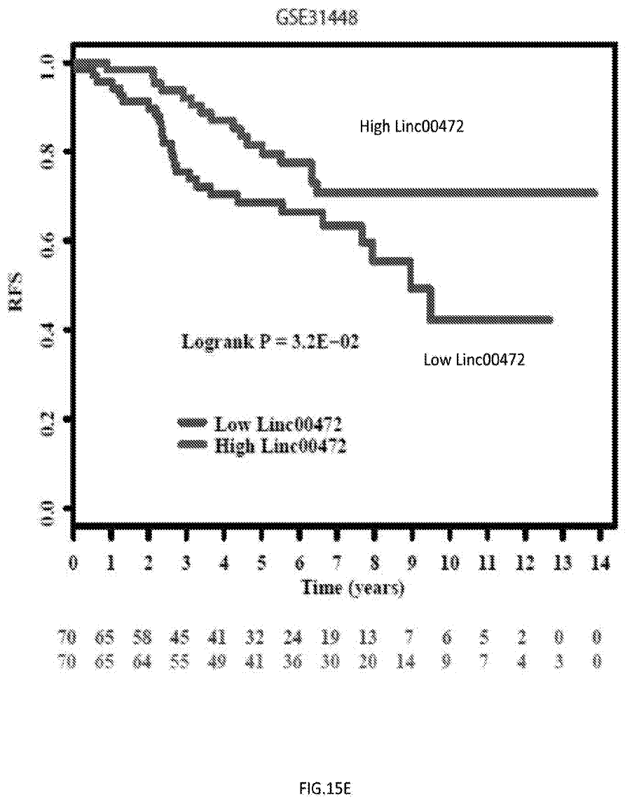

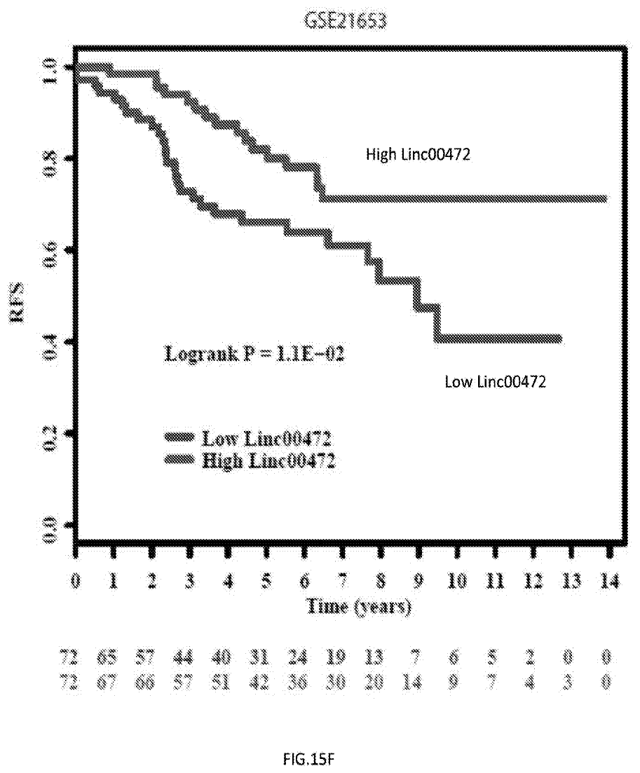

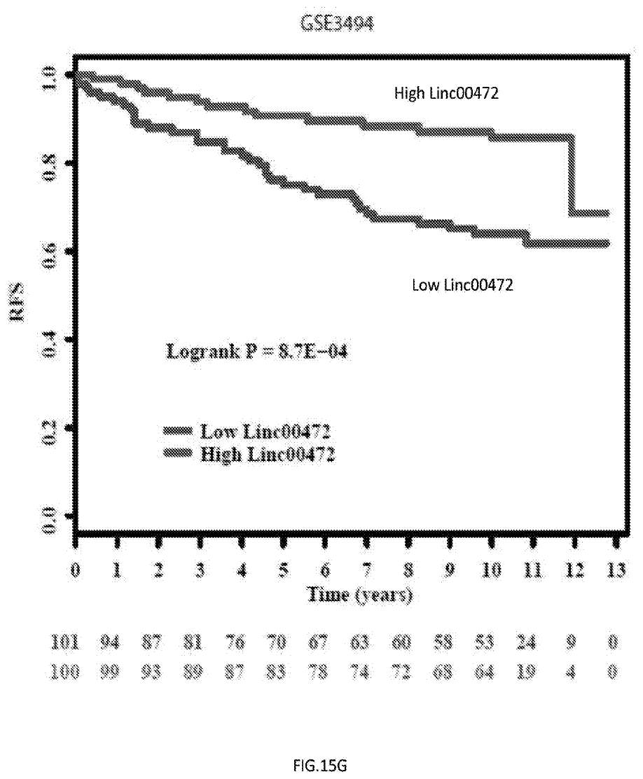

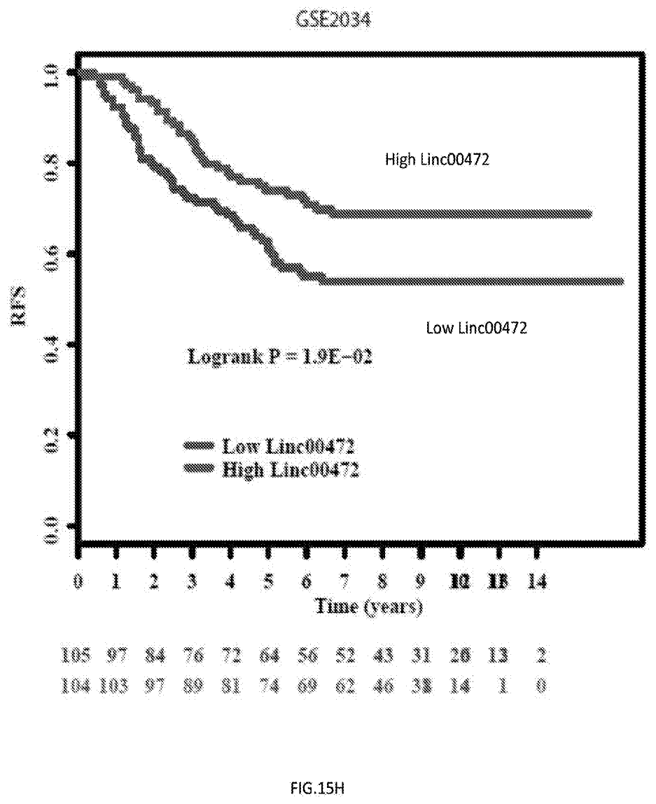

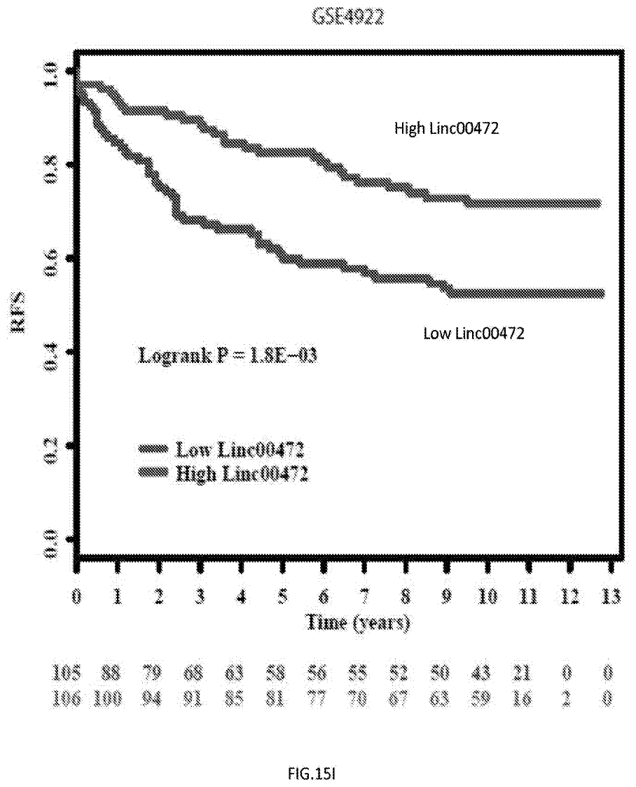

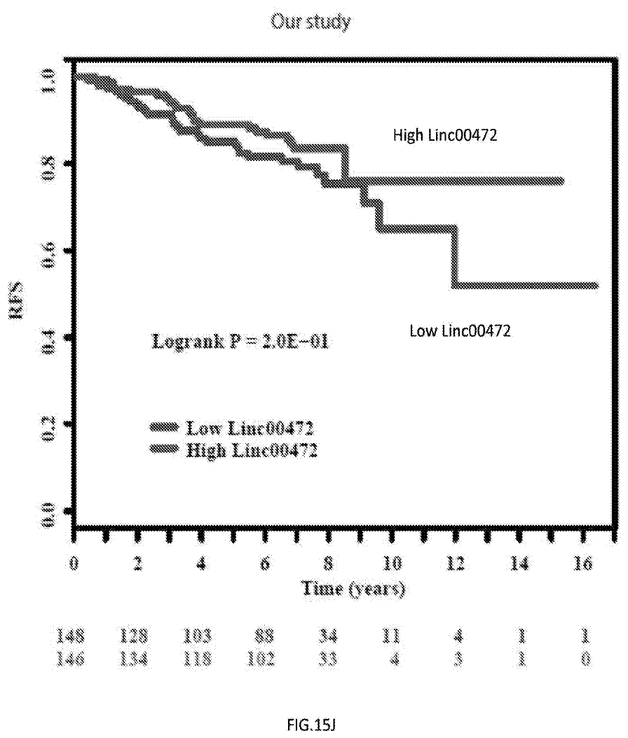

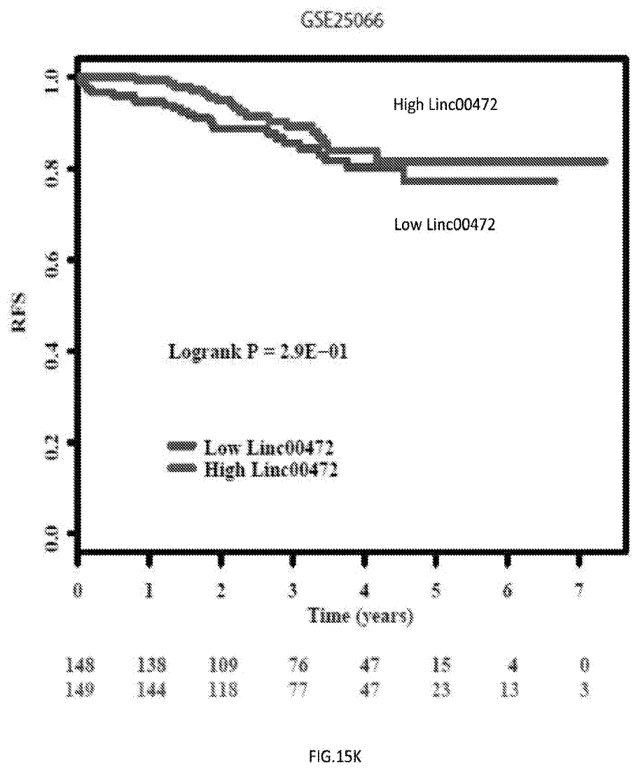

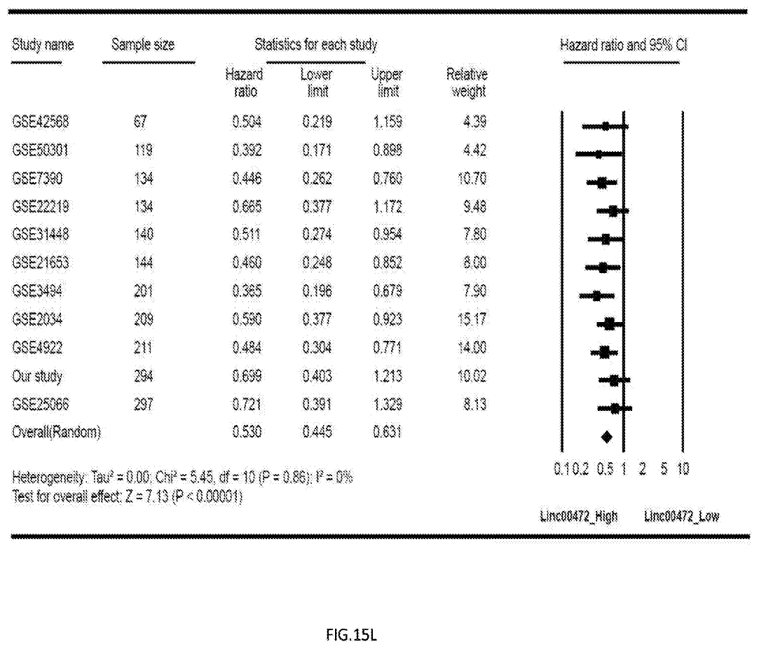

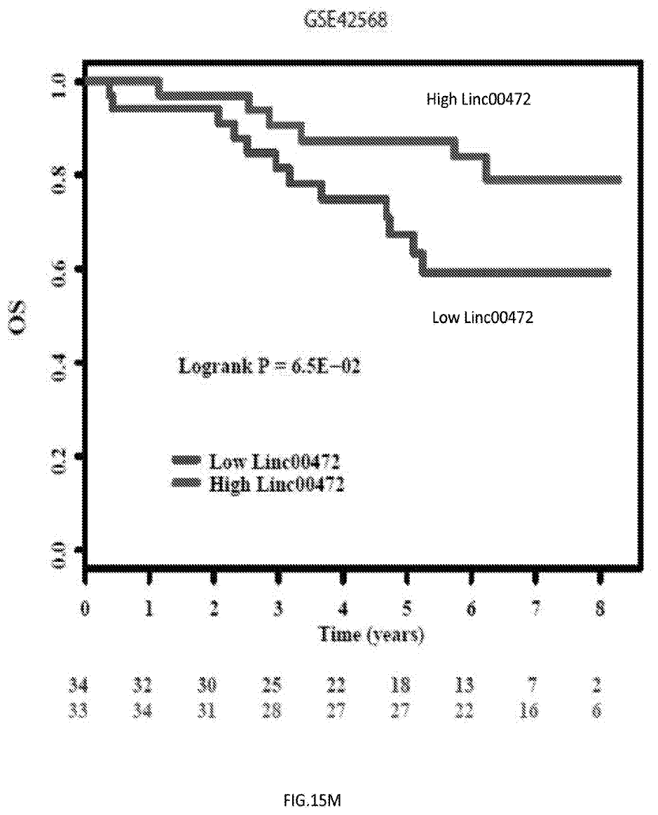

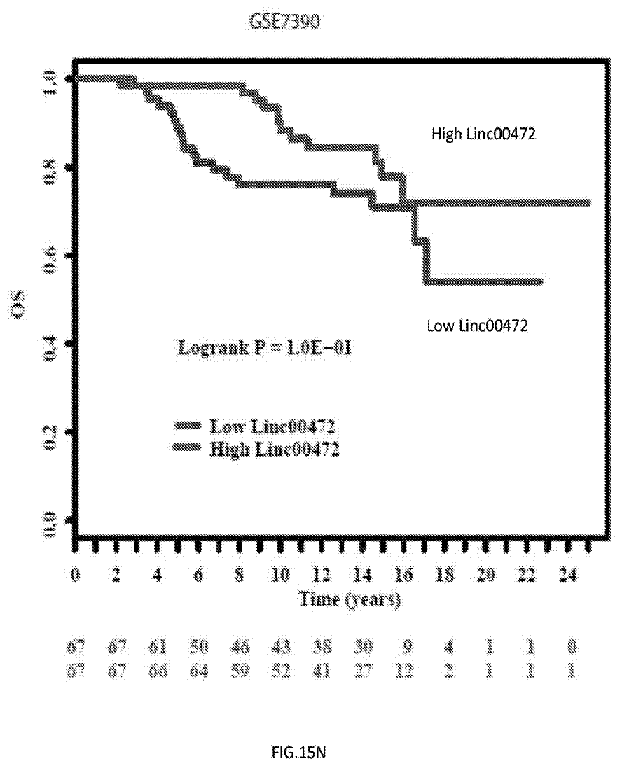

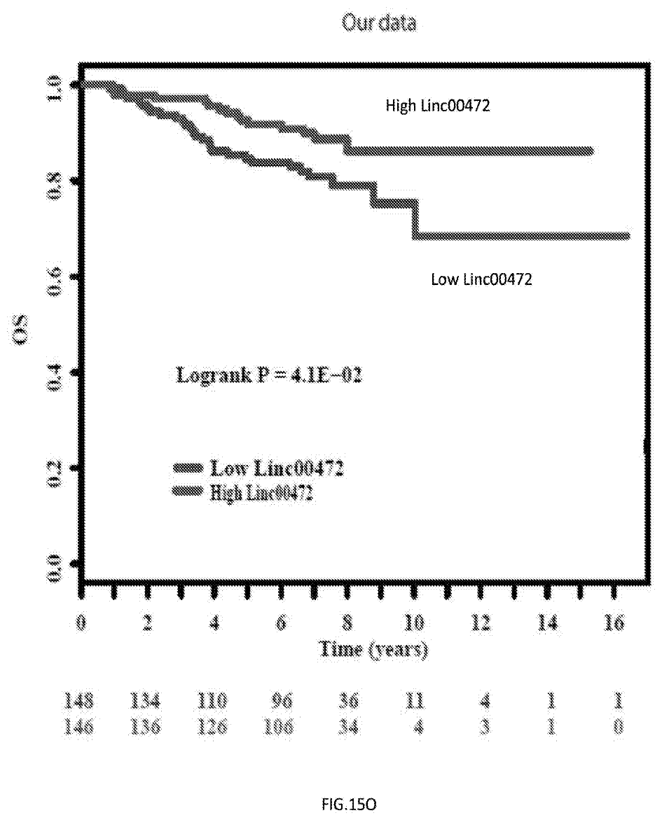

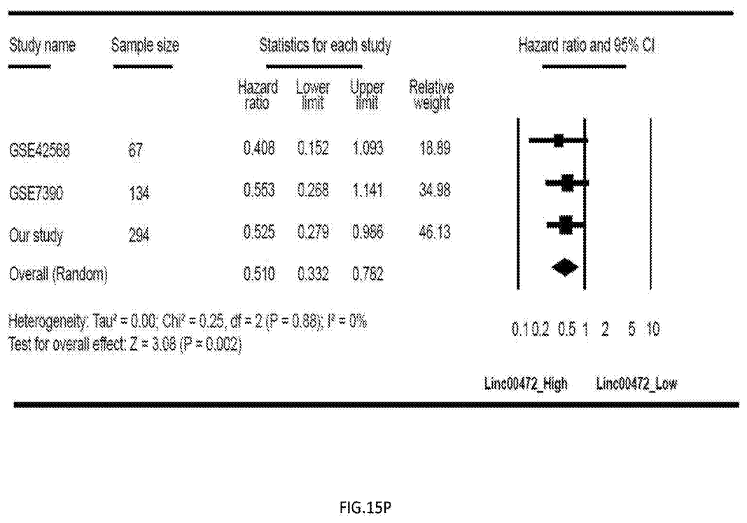

Higher LINC00472 expression was shown in ER-positive than in ER-negative tumors. The summarized odds ratio for high LINC00472 in ER-negative tumors was low, only 0.425. ER-positive patients with high LINC00472 had better relapse-free survival (as shown in FIGS. 15A-13P) and overall survival (as shown in FIGS. 15M-15O) compared to ER-positive patients with low LINC00472. The risk for relapse or death was reduced by more than 40% (as shown in FIGS. 15L and 15P).

The above test results confirm that high expression of LINC00472 is associated with favorable survival outcomes of breast cancer. In particular, according to the present invention, stable transfection of LINC00472 were made in MB231 and Hs578T, two triple-negative cell lines. As a result, LINC00472 overexpression suppressed not only cell proliferation and migration, but also cell invasion and colony formation. The inhibitory effect of LINC00472 on tumor growth was also observed in vivo using a xenograft mouse model.

As illustrated above, to illustrate the molecular mechanism, the transcriptomes and metabolomes of aggressive breast cancer cells with LINC00472 overexpression were also analyzed. Down-regulation of TNF-α was suggested in the expression profiles of both cell lines. Based on the bioinformatics, it was surprisingly found that NF-κB, a downstream target of the TNF-α signaling, was affected by the lncRNA, and LINC00472 inhibited the phosphorylation of IκBα and p65. The data on metabolome also indicated the involvement of LINC00472 in NF-κB because the lncRNA was predicted to have an impact on methionine metabolism. In vitro experiment on several tumor cell lines of the central nervous system showed that methionine restriction inhibited nuclear translocation of NF-κB. The expression profiles of MB231 and Hs578T with LINC00472 overexpression suggested ESR1 being a possible upstream signal for LINC00472. Based on the gene sequence, an ERα binding site was predicted in the LINC00472 promoter, and the prediction was confirmed by in vitro experiments, which showed that ERα was able to bind to the predicted region, upregulating LINC00472 expression. These results were further supported by the experiments on an ER-positive cell line, T47D, in which LINC00472 expression was high and suppressing ER by tamoxifen could lower the expression of LINC00472. Data analysis of multiple clinical studies including our own also showed that high expression of LINC00472 was associated with ER-positive tumors and favorable prognosis in ER-positive patients. Furthermore, the experiments demonstrated that suppression of NF-κB by ERα was mediated through LINC00472 and blocking the expression of LINC00472 could release the inhibitory effect of ERα on NF-κB.

All these findings show that LINC00472 plays an important role in NF-κB and ERα interaction which has been implicated in the development of endocrine resistance in ER-positive breast cancer.

Approximately a third ER-positive tumors develop endocrine resistance to tamoxifen treatment. By far, several possible mechanisms have been proposed by researches for this phenomenon, one of which is the interaction between ERα and NF-κB. Estradiol could reduce inflammation by suppressing the DNA-binding ability of NF-κB, or blocking the translocation of NF-κB from cytoplasm to nucleus. Inhibition of NF-κB by estrogen was observed in several studies. In addition, NF-κB was found to suppress estrogen. NF-κB could inhibit the activity of ERα or its expression. Analysis of tumor samples indicated mutual inhibition between ERα and NF-κB. Although much evidence suggests an antagonistic interaction, a few studies reported a synergy between ERα and NF-κB. NF-κB was known to play an important role in the progression of ER-negative tumors. It was also found that ER-positive patients with high NF-κB activities could develop resistance to tamoxifen compared to ER-positive patients with low NF-κB.

Furthermore, ER-positive tumor cells with high NF-κB expression was known to be linked to no response to tamoxifen, and inhibition of NF-κB could reduce tumor cell's resistance to endocrine treatment. Prior studies demonstrated that the development of tamoxifen resistance was accompanied by increased NF-κB activities. These observations may indicate that ER suppression by endocrine therapy may release its inhibition on NF-κB, resulting in rising NF-κB activities that promote tumor growth and invasion, leading to disease recurrence and tumor metastasis. In agreement with this speculation, the experiments—according to the present invention—on T47D showed that tamoxifen treatment of this ER-positive tumor cell line resulted in suppression of ERα which led to increases in phosphorylation of IκBα and p65 and reduction in LINC00472. According to the present invention, the surprising discovery of LINC00472's connection to ERα and NF-κB in tumor cells as well as observation of associations between LINC00472 expression and ER status and disease outcomes in breast cancer patients provide new insights into the relationship between ERα and NF-κB and their role in endocrine resistance. Accordingly, ER-positive tumors treated with tamoxifen are initially responsive to the treatment when estrogen-stimulated cell proliferation is blocked by ERα suppression, but this blockage also suppresses LINC00472, which releases its inhibition on NF-κB, resulting in increases in NF-κB activities that facilitate tumor growth and metastasis. The increase in NF-κB activities may also help to select tumor clones that are not sensitive to ERα in growth regulation.

In summary, increasing LINC00472 expression in breast tumor cells reduces aggressive cell behaviors and suppresses tumor growth. ERα binds to the LINC00472 promoter, up-regulating its expression. LINC00472 inhibits the activity of NF-κB and mediates the inhibitory effect of ERα on NF-κB. High expression of LINC00472 was associated with ER-positive breast tumors and favorable survival of ER-positive patients. Tamoxifen treatment of ER-positive tumor cells suppresses ERα and LINC00472 which results in an elevated activity of NF-κB. Taken together, long-term use of tamoxifen may release LINC00472's inhibition on NF-κB, leading to endocrine resistance and tumor recurrence. As such, regulation of LINC00472 in breast cancer according to the present invention may help to address the challenge of endocrine resistance in breast cancer treatment.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this invention pertains.

It is to be understood that any variations evident to one of ordinary skill in the art also fall within the scope of the claimed invention and thus, the selection of specific component elements can be determined without departing from the spirit of the invention herein disclosed and described. Furthermore, the present invention is not to be limited to the examples and embodiments set forth herein, which are only intended to illustrate the present invention. Any combination of topical systems, cosmetic patches, fabrication processes, and applications of this invention, along with any obvious their extension or analogs, are within the scope of this invention. Further, it is intended that this invention encompass any arrangement, which is calculated to achieve that same purpose, and all such variations and modifications as fall within the scope of the appended claims.

1. A method of determining or predicting the resistance of a cancer cell in a biological subject to a treatment, comprising:
(i) detecting an expression of RNA linc00472 in a sample obtained from the biological subject;
(ii) comparing the detected expression with a control; and
(iii) determining or predicting the resistance of the cancer to the treatment based on the comparison.

2. The method of claim 1, wherein the treatment inhibits ERα pathway in the biological subject.

3. The method of claim 1, wherein the treatment elevates the activity of NF-κB pathway in the biological subject.

4. The method of claim 1, wherein a higher detected level of RNA linc00472 as compared to the control indicates a better relapse-free survivor or overall survival.

5. The method of claim 1, wherein the treatment is an endocrine therapy.

6. The method of claim 1, wherein the cancer is breast cancer.

7. A kit for diagnosing or treating a cancer or predisposition thereto in a biological subject, or determining the resistance of the cancer to a treatment, comprising (i) a biomarker that detects the level of RNA linc00472 in a sample obtained from the biological subject; and (ii) a control, to be compared with the detected level.

8. The kit of claim 7, wherein a higher detected level of RNA linc00472 as compared to the control indicates the presence of the cancer or predisposition thereto.

9. The kit of claim 8, wherein the cancer is estrogen receptor-positive.

10. The kit of claim 9, wherein the cancer is breast cancer.

11. The kit of claim 7, wherein the treatment is an endocrine therapy.

12. The kit of claim 11, wherein a higher detected level of RNA linc00472 as compared to the control indicates a better relapse-free survivor or overall survival than a predetermined survivor rate.

13. A method of treating a cancer or predisposition thereto, in a biological subject, comprising administration to the biological subject in need thereof of an effective amount of a NF-κB pathway inhibitor.

14. The method of claim 13, wherein the NF-κB pathway inhibitor upregulates a level of RNA linc00472 in the biological subject.

15. The method of claim 14, wherein the NF-κB pathway inhibitor comprises a linc00472-expressing plasmid or vector.

16. A method of inhibiting an activity of NF-κB pathway in a biological subject, comprising administration of an effective amount of a composition that upregulates the level of RNA linc00472, to the biological subject.

17. The method of claim 16, wherein the composition comprises a linc00472-expressing plasmid and a pharmaceutically acceptable carrier.

18. (canceled)

19. The method of claim 17, wherein the composition further comprises a transfection agent to facilitate the delivery of RNA linc00472 to the cells.

20. A method of preventing the resistance of a cancer cell in a biological subject to a treatment, comprising administration of an effective amount of a compound that upregulates the level of RNA linc00472 in a sample.

21. The method of claim 20, wherein the compound comprises a linc00472-expressing plasmid or vector.

22. (canceled)
